(19) United States
(12) Patent Application Publication
(10) Pub. No.: US 2020/0085832 A1
(43) Pub. Date: Mar. 19, 2020

(54) DEUTERATED ANALOGS OF AN ORGANIC COMPOUND

(71) Applicant: Agios Pharmaceuticals, Inc., Cambridge, MA (US)

(72) Inventors: Zhihua Sui, Somerville, MA (US); Robert Zahler, Pennington, NJ (US)

(21) Appl. No.: 16/692,217

(22) Filed: Nov. 22, 2019

(51) Int. Cl. A61K 31/53; A61K 45/06; C07D 401/04; A61P 35/00; A61N 5/10

(52) U.S. Cl. CPC A61K 31/53; A61K 45/06; A61N 5/10; A61P 35/00; C07D 401/04

(57) ABSTRACT

Provided are deuterated analogs of a compound and methods of using such deuterated analogs for treating a brain tumor in a patient in need thereof; the treatment comprising administering to the patient a deuterated compound described herein. The deuterated compound may be administered in combination with radiation therapy and/or an additional therapeutic agent.

### BACKGROUND

Isocitrate dehydrogenases (IDHs) catalyze the oxidative decarboxylation of isocitrate to 2-oxoglutarate (i.e., α-ketoglutarate). These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

IDH1 (isocitrate dehydrogenase 1 (NADP+), cytosolic) is also known as IDH; IDP; IDCD; IDPC or PICD. The protein encoded by this gene is the NADP(+)-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2,4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production.

The human IDH1 gene encodes a protein of 414 amino acids. The nucleotide and amino acid sequences for human IDH1 can be found as GenBank entries NM_005896.2 and NP_005887.2 respectively. The nucleotide and amino acid sequences for IDH1 are also described in, e.g., Nekrutenko et al., Mol. Biol. Evol. 15:1674-1684(1998); Geisbrecht et al., J. Biol. Chem. 274:30527-30533(1999); Wiemann et al., Genome Res. 11:422-435(2001); The MGC Project Team, Genome Res. 14:2121-2127(2004); Lubec et al., Submitted (December 2008) to UniProtKB; Kullmann et al., Submitted (June 1996) to the EMBL/GenBank/DDBJ databases; and Sjoeblom et al., Science 314:268-274(2006).

Non-mutant, e.g., wild type, IDH1 catalyzes the oxidative decarboxylation of isocitrate to α-ketoglutarate.

It has been discovered that mutations of IDH1 present in certain cancer cells result in a new ability of the enzyme to catalyze the NADPH-dependent reduction of α-ketoglutarate to R(−)-2-hydroxyglutarate (2HG). The production of 2HG is believed to contribute to the formation and progression of cancer (Dang, L et al., Nature 2009, 462:739-44).

IDH2 (isocitrate dehydrogenase 2 (NADP+), mitochondrial) is also known as IDH; IDP; IDHM; IDPM; ICD-M; or mNADP-IDH. The protein encoded by this gene is the NADP(+)-dependent isocitrate dehydrogenase found in the mitochondria. It plays a role in intermediary metabolism and energy production. This protein may tightly associate or interact with the pyruvate dehydrogenase complex. Human IDH2 gene encodes a protein of 452 amino acids. The nucleotide and amino acid sequences for IDH2 can be found as GenBank entries NM_002168.2 and NP_002159.2 respectively. The nucleotide and amino acid sequence for human IDH2 are also described in, e.g., Huh et al., Submitted (November 1992) to the EMBL/GenBank/DDBJ databases; and The MGC Project Team, Genome Res. 14:2121-2127(2004).

Non-mutant, e.g., wild type, IDH2 catalyzes the oxidative decarboxylation of isocitrate to α-ketoglutarate (α-KG).

It has been discovered that mutations of IDH2 present in certain cancer cells result in a new ability of the enzyme to catalyze the NADPH-dependent reduction of α-ketoglutarate to R(−)-2-hydroxyglutarate (2HG). 2HG is not formed by wild-type IDH2. The production of 2HG is believed to contribute to the formation and progression of cancer (Dang, L et al, Nature 2009, 462:739-44).

Mutations in IDH1 or IDH2 occur in over 70% of diffuse low grade glioma (LGG) tumors. IDH mutations result in accumulation of 2-HG, which is believed to facilitate tumorigenesis through DNA hypermethylation, increased repressive histone methylation, and inhibition of differentiation processes. Studies performed with a tool compound known as AGI-5198, which has been shown to inhibit mutant IDH1 (mIDH1), but not mutant IDH2 (mIDH2), have demonstrated that inhibition of mIDH1 proteins can repress growth of mIDH1-driven gliomas in some model systems (D. Rohle et al. Science 340:626-630 (2013)).

U.S. Publication No. 2015/0018328 A1 discloses a compound described by the chemical name 6-(6-chloropyridin-2-yl)-N²,N⁴-bis((R)-1,1,1-trifluoropropan-2-yl)-1,3,5-triazine-2,4-diamine (Compound X), which has been shown to act as an inhibitor of mutant IDH1 and IDH2 proteins in biochemical and cellular assays. This application, inter alia, describes various analogs of Compound X that incorporate one or more deuterium atoms and the use of such deuterated analogs in the treatment of brain tumors.

Given that the effect of deuteration is unpredictable, variability in deuterium effects has also led experts to question or dismiss deuterium modification as a viable drug design strategy for inhibiting adverse metabolism (see Foster, A B, Adv Drug Res 1985, 14:1-40 and Fisher, M B et al., Curr Opin Drug Discov Devel, 2006, 9:101-09). However, a potentially attractive strategy for improving a drug's metabolic properties is deuterium modification, especially at the metabolic sites of the drug. In this approach, one attempts to slow the cyp-mediated metabolism of a drug or to reduce the formation of undesirable metabolites by replacing one or more hydrogen atoms with deuterium atoms at the metabolic site of the drug. Deuterium is a safe, stable, non-radioactive isotope of hydrogen. Compared to hydrogen, deuterium forms slightly stronger bonds with carbon, which may impact pharmacokinetics of a drug with the potential for improved drug efficacy, safety, and/or tolerability, without affecting the biochemical potency and selectivity of the drug as compared to non-deuterated analog. This application provides for compounds that should have enhanced drug efficacy, safety, and/or tolerability in which deuterium atoms are introduced at various carbon atoms.

### SUMMARY

This application provides various deuterated analogs of Compound X, specifically compounds of formula (I), and pharmaceutically acceptable salts or pharmaceutically acceptable co-crystalline materials thereof. Also provided are compositions comprising such deuterated analogs or their pharmaceutically acceptable salts or pharmaceutically acceptable co-crystalline materials, as well as methods of using such deuterated analogs or their pharmaceutically acceptable salts or pharmaceutically acceptable co-crystalline materials.

In one aspect, the application provides a compound of formula (I)

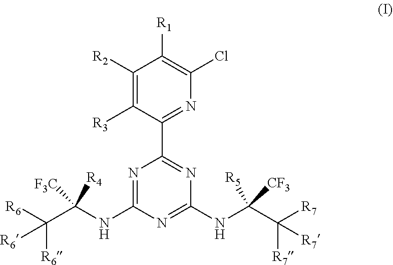

wherein each of R₁, R₂, R₃, R₄, R₅, R₆, R_{6′}, R_{6″}, R₇, R_{7′}, and R_{7″} is independently H or D provided that at least one of R₁, R₂, R₃, R₄, R₅, R₆, R_{6′}, R_{6″}, R₇, R_{7′}, and R_{7″} is D, or a pharmaceutically acceptable salt or a pharmaceutically acceptable co-crystalline material thereof, where D represents a deuterium atom.

In another aspect, the application provides a pharmaceutical composition comprising a compound of formula (I)

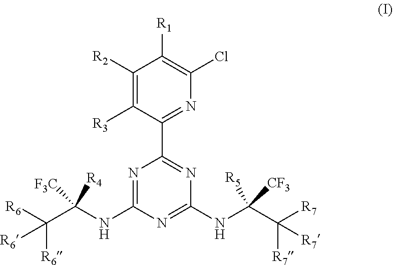

wherein each of R₁, R₂, R₃, R₄, R₅, R₆, R_{6′}, R_{6″}, R₇, R_{7′}, and R_{7″} is independently H or D provided that at least one of R₁, R₂, R₃, R₄, R₅, R₆, R_{6′}, R_{6″}, R₇, R_{7′}, and R_{7″} is D, or a pharmaceutically acceptable salt or a pharmaceutically acceptable co-crystalline material thereof and one or more pharmaceutically acceptable excipients, where D represents a deuterium atom.

In another aspect, the application provides a method for treating a brain tumor characterized by the presence of an IDH1 and/or IDH2 mutation in a patient in need thereof comprising administering to the patient a compound of formula (I)

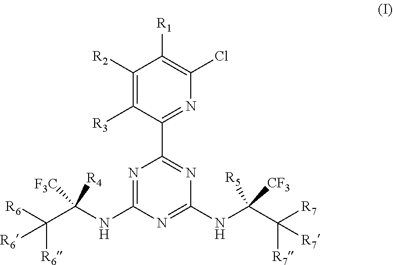

wherein each of R₁, R₂, R₃, R₄, R₅, R₆, R_{6′}, R_{6″}, R₇, R_{7′}, and R_{7″} is independently H or D provided that at least one of R₁, R₂, R₃, R₄, R₅, R₆, R_{6′}, R_{6″}, R₇, R_{7′}, and R_{7″} is D, or a pharmaceutically acceptable salt or a pharmaceutically acceptable co-crystalline material thereof, where D represents a deuterium atom.

In another aspect, the application provides a method for treating a brain tumor characterized by the presence of an IDH1 and/or IDH2 mutation in a patient in need thereof comprising administering to the patient (a) a compound of formula (I)

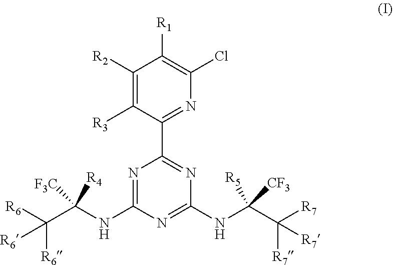

wherein each of R₁, R₂, R₃, R₄, R₅, R₆, R_{6′}, R_{6″}, R₇, R_{7′}, and R_{7″} is independently H or D provided that at least one of R₁, R₂, R₃, R₄, R₅, R₆, R_{6′}, R_{6″}, R₇, R_{7′}, and R_{7″} is D, or a pharmaceutically acceptable salt or a pharmaceutically acceptable co-crystalline material thereof, where D represents a deuterium atom; and (b) radiation therapy; in amounts effective for treating the brain tumor.

In another aspect, the application provides a method for treating a brain tumor characterized by the presence of an IDH1 and/or IDH2 mutation in a patient in need thereof comprising administering to the patient (a) a compound of formula (I)

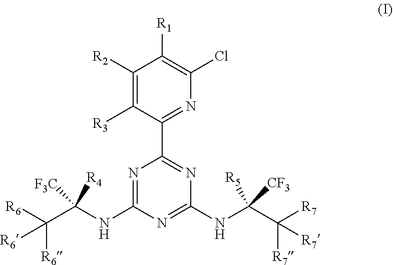

wherein each of R₁, R₂, R₃, R₄, R₅, R₆, R_{6′}, R_{6″}, R₇, R_{7′}, and R_{7″} is independently H or D provided that at least one of R₁, R₂, R₃, R₄, R₅, R₆, R_{6′}, R_{6″}, R₇, R_{7′}, and R_{7″} is D, or a pharmaceutically acceptable salt or a pharmaceutically acceptable co-crystalline material thereof, where D represents a deuterium atom; (b) an additional therapeutic agent; and optionally (c) radiation therapy; in amounts effective for treating the brain tumor.

### DETAILED DESCRIPTION

In one aspect, the application provides a compound of formula (I)

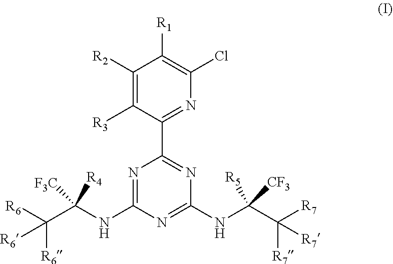

wherein each of R₁, R₂, R₃, R₄, R₅, R₆, R_{6′}, R_{6″}, R₇, R_{7′}, and R_{7″} is independently H or D provided that at least one of the R groups is D, or a pharmaceutically acceptable salt or a pharmaceutically acceptable co-crystalline material thereof, where D represents a deuterium atom.

In one embodiment, each of R₁, R₂, R₃, R₄, R₅, R₆, R_{6′}, R_{6″}, R₇, R_{7′}, and R_{7″} is D.

In another embodiment, any one or two or all three of R₁, R₂, or R₃ are D and each of R₄, R₅, R₆, R_{6′}, R_{6″}, R₇, R_{7′}, and R_{7″} is H. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, one or two or all three of R₁, R₂, or R₃ is or are D and either or both of R₄ and R₅ is or are D and each of R₆, R_{6′}, R_{6″}, R₇, R_{7′}, and R_{7″} is H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and either or both of R₄ and R₅ is or are D and all three of R₆, R_{6′}, and R_{6″} are D and each of R₇, R_{7′}, and R_{7″} is H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or R₄ is H and R₅ is D or both R₄ and R₅ are D.

In another embodiment, one or two or all three of R₁, R₂, and R₃ is or are D and both of R₄ and R₅ are H and each of R₆, R_{6′}, and R_{6″} is D and each of R₇, R_{7′}, and R_{7″} is H. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, one or two or all three of R₁, R₂, and R₃ is or are D and either or both of R₄ and R₅ is or are D and each of R₆, R_{6′}, R_{6″}, R₇, R_{7′}, and R_{7″} is D. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and both of R₄ and R₅ are H and each of R₆, R_{6′}, R_{6″}, R₇, R_{7′}, and R_{7″} is D. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, all three of R₁, R₂, and R₃ are H and each of R₄, R₅, R₆, R_{6′}, R_{6″}, R₇, R_{7′}, and R_{7″} is D.

In another embodiment, all three of R₁, R₂, and R₃ are H and either of R₄ and R₅ is D and each of R₆, R_{6′}, R_{6″}, R₇, R_{7′}, and R_{7″} is D. In some of such embodiments, R₄ is H and R₅ is D.

In another embodiment, all three of R₁, R₂, and R₃ are H and either or both of R₄ and R₅ is or are D and all three of R₆, R_{6′}, and R_{6″} are D and all three of R₇, R_{7′}, and R_{7″} are H. In some of such embodiments, R₄ is H and R₅ is D. In other of such embodiments, R₄ is D and R₅ is H. In still other of such embodiments, both of R₄ and R₅ are D.

In another embodiment, all three of R₁, R₂, and R₃ are H and either or both of R₄ and R₅ is or are D and each of R₆, R_{6′}, R_{6″}, R₇, R_{7′} and R_{7″} is H. In some of such embodiments, R₄ is H and R₅ is D. In still other of such embodiments, both of R₄ and R₅ are D.

In another embodiment, all three of R₁, R₂, and R₃ are H and both of R₄ and R₅ are H and each of R₆, R_{6′}, R_{6″}, R₇, R_{7′} and R_{7″} is D.

In another embodiment, all three of R₁, R₂, and R₃ are H and both of R₄ and R₅ are H and each of R₆, R_{6′}, and R_{6″} is D and each of R₇, R_{7′}, and R_{7″} is H.

In still other embodiments of compounds of Formula I, at least one of R₆, R_{6′}, R_{6″}, R₇, R_{7′}, and R_{7″} is D. In some of such embodiments, any two or three or four or five of R₆, R_{6′}, R_{6″}, R₇, R_{7′}, and R_{7″} are D. In other such embodiments, any two of R₆, R_{6′}, R_{6″}, R₇, R_{7′}, and R_{7″} are D. In other such embodiments, any three of R₆, R_{6′}, R_{6″}, R₇, R_{7′}, and R_{7″} are D. In other such embodiments, any four of R₆, R_{6′}, R_{6″}, R₇, R_{7′}, and R_{7″} are D. In other such embodiments, any five of R₆, R_{6′}, R_{6″}, R₇, R_{7′}, and R_{7″} are D.

In one aspect, the application provides a pharmaceutical composition comprising a compound of formula (I)

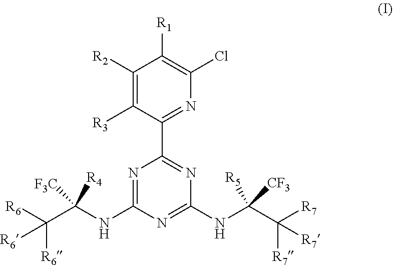

wherein each of R₁, R₂, R₃, R₄, R₅, R₆, R_{6′}, R_{6″}, R₇, R_{7′}, and R_{7″} is independently H or D provided that at least one of the R groups is D, or a pharmaceutically acceptable salt thereof or a pharmaceutically acceptable co-crystalline material and one or more pharmaceutically acceptable excipients, where D represents a deuterium atom.

In one embodiment, each of R₁, R₂, R₃, R₄, R₅, R₆, R_{6′}, R_{6″}, R₇, R_{7′}, and R_{7″} is D.

In another embodiment, any one or two or all three of R₁, R₂, or R₃ is or are D and each of R₄, R₅, R₆, R_{6′}, R_{6″}, R₇, R_{7′}, and R_{7″} is H. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, one or two or all three of R₁, R₂, or R₃ is or are D and either or both of R₄ and R₅ is or are D and each of R₆, R_{6′}, R_{6″}, R₇, R_{7′}, and R_{7″} is H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and either or both of R₄ and R₅ is or are D and all three of R₆, R_{6′}, and R_{6″} are D and all three of R₇, R_{7′}, and R_{7″} are H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or R₄ is H and R₅ is D or both R₄ and R₅ are D.

In another embodiment, one or two or all three of R₁, R₂, and R₃ is or are D and both of R₄ and R₅ are H and each of R₆, R_{6′}, and R_{6″} is D and each of R₇, R_{7′}, and R_{7″} is H.

In some of such embodiments, any one of R₁, R₂, or R₃ is or are D. In other such embodiments, any two of R₁, R₂, or R₃ are D. In still another of such embodiments, all three of R₁, R₂, and R₃ are D.

In another embodiment, one or two or all three of R₁, R₂, and R₃ is or are D and either or both of R₄ and R₅ is or are D and each of R₆, R_{6′} R_{6″}, R₇, R_{7′}, and R_{7″} is D. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and both of R₄ and R₅ are H and each of R₆, R_{6′}, R_{6″}, R₇, R_{7′}, and R_{7″} is D. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, all three of R₁, R₂, and R₃ are H and each of R₄, R₅, R₆, R_{6′}, R_{6″}, R₇, R_{7′}, and R_{7″} is D.

In another embodiment, all three of R₁, R₂, and R₃ are H and either of R₄ and R₅ is or are D and each of R₆, R_{6′}, R_{6″}, R₇, R_{7′}, and R_{7″} is D. In some of such embodiments, R₄ is H and R₅ is D.

In another embodiment, all three of R₁, R₂, and R₃ are H and either or both of R₄ and R₅ is or are D and all three of R₆, R_{6′}, and R_{6″} are D and all three of R₇, R_{7′}, and R_{7″} are H. In some of such embodiments, R₄ is H and R₅ is D. In other of such embodiments, R₄ is D and R₅ is H. In still other of such embodiments, both of R₄ and R₅ are D.

In another embodiment, all three of R₁, R₂, and R₃ are H and either or both of R₄ and R₅ is or are D and each of R₆, R_{6′}, R_{6″}, R₇, R_{7′}, and R_{7″} is H. In some of such embodiments, R₄ is H and R₅ is D. In still other of such embodiments, both of R₄ and R₅ are D.

In another embodiment, all three of R₁, R₂, and R₃ are H and both of R₄ and R₅ are H and each of R₆, R_{6′}, R_{6″}, R₇, R_{7′}, and R_{7″} is D.

In another embodiment, all three of R₁, R₂, and R₃ are H and both of R₄ and R₅ are H and each of R₆, R_{6′}, and R_{6″} is D and each of R₇, R_{7′}, and R_{7″} is H.

In still other embodiments of compounds of Formula I, at least one of R₆, R_{6′}, R_{6″}, R₇, R_{7′}, and R_{7″} is D. In some of such embodiments, any two or three or four or five of R₆, R_{6′}, R_{6″}, R₇, R_{7′}, and R_{7″} are D. In other such embodiments, any two of R₆, R_{6′}, R_{6″}, R₇, R_{7′}, and R_{7″} are D. In other such embodiments, any three of R₆, R_{6′}, R_{6″}, R₇, R_{7′}, and R_{7″} are D.

In other such embodiments, any four of R₆, R_{6′}, R_{6″}, R₇, R_{7′}, and R_{7″} are D. In other such embodiments, any five of R₆, R_{6′}, R_{6″}, R₇, R_{7′}, and R_{7″} are D.

In another aspect, the application provides a method for treating a brain tumor characterized by the presence of an IDH1 and/or IDH2 mutation in a patient in need thereof comprising administering to the patient a compound of formula (I) or a pharmaceutical composition comprising a compound of formula (I)

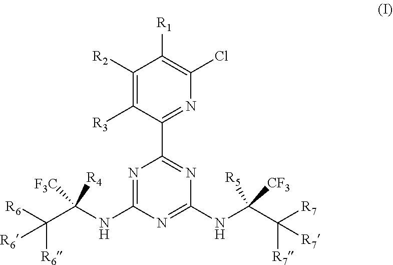

wherein each of R₁, R₂, R₃, R₄, R₅, R₆, R_{6′}, R_{6″}, R₇, R_{7′} and R_{7″} is independently H or D provided that at least one of the R₁, R₂, R₃, R₄, R₅, R₆, R_{6′}, R_{6″}, R₇, R_{7′} and R_{7″} is D, or a pharmaceutically acceptable salt or a pharmaceutically acceptable co-crystalline material thereof, in amounts effective for treating the brain tumor, where D represents a deuterium atom.

In one embodiment, each of R₁, R₂, R₃, R₄, R₅, R₆, R_{6′}, R_{6″}, R₇, R_{7′}, and R_{7″} is D.

In another embodiment, any one or two or all three of R₁, R₂, or R₃ is or are D and each of R₄, R₅, R₆, R_{6′}, R_{6″}, R₇, R_{7′}, and R_{7″} is H. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, one or two or all three of R₁, R₂, or R₃ is or are D and either or both of R₄ and R₅ is or are D and each of R₆, R_{6′}, R_{6″}, R₇, R_{7′}, and R_{7″} is H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and either or both of R₄ and R₅ is or are D and all three of R₆, R_{6′}, and R_{6″} are D and all three of R₇, R_{7′}, and R_{7″} are H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or R₄ is H and R₅ is D or both R₄ and R₅ are D.

In another embodiment, one or two or all three of R₁, R₂, and R₃ is or are D and both of R₄ and R₅ are H and each of R₆, R_{6′}, and R_{6″} is D and each of R₇, R_{7′}, and R_{7″} is H. In some of such embodiments, any one of R₁, R₂, or R₃ is or are D. In other such embodiments, any two of R₁, R₂, or R₃ are D. In still another of such embodiments, all three of R₁, R₂, and R₃ are D.

In another embodiment, one or two or all three of R₁, R₂, and R₃ is or are D and either or both of R₄ and R₅ is or are D and each of R₆, R_{6′} R_{6″}, R₇, R_{7′}, and R_{7″} is D. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and both of R₄ and R₅ are H and each of R₆, R_{6′}, R_{6″}, R₇, R_{7′}, and R_{7″} is D. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, all three of R₁, R₂, and R₃ are H and each of R₄, R₅, R₆, R_{6′}, R_{6″}, R₇, R_{7′}, and R_{7″} is D.

In another embodiment, all three of R₁, R₂, and R₃ are H and either of R₄ and R₅ is or are D and each of R₆, R_{6′}, R_{6″}, R₇, R_{7′}, and R_{7″} is D. In some of such embodiments, R₄ is H and R₅ is D.

In another embodiment, all three of R₁, R₂, and R₃ are H and either or both of R₄ and R₅ is or are D and all three of R₆, R_{6′}, and R_{6″} are D and all three of R₇, R_{7′}, and R_{7″} are H. In some of such embodiments, R₄ is H and R₅ is D. In other of such embodiments, R₄ is D and R₅ is H. In still other of such embodiments, both of R₄ and R₅ are D.

In another embodiment, all three of R₁, R₂, and R₃ are H and either or both of R₄ and R₅ is or are D and each of R₆, R_{6′}, R_{6″}, R₇, R_{7′}, and R_{7″} is H. In some of such embodiments, R₄ is H and R₅ is D. In still other of such embodiments, both of R₄ and R₅ are D.

In another embodiment, all three of R₁, R₂, and R₃ are H and both of R₄ and R₅ are H and each of R₆, R_{6′}, R_{6″}, R₇, R_{7′}, and R_{7″} is D.

In another embodiment, all three of R₁, R₂, and R₃ are H and both of R₄ and R₅ are H and each of R₆, R_{6′}, and R_{6″} is D and each of R₇, R_{7′}, and R_{7″} is H.

In still other embodiments of compounds of Formula I, at least one of R₆, R_{6′}, R_{6″}, R₇, R_{7′}, and R_{7″} is D. In some of such embodiments, any two or three or four or five of R₆, R_{6′}, R_{6″}, R₇, R_{7′}, and R_{7″} are D. In other such embodiments, any two of R₆, R_{6′}, R_{6″}, R₇, R_{7′}, and R_{7″} are D. In other such embodiments, any three of R₆, R_{6′}, R_{6″}, R₇, R_{7′}, and R_{7″} are D. In other such embodiments, any four of R₆, R_{6′}, R_{6″}, R₇, R_{7′}, and R_{7″} are D. In other such embodiments, any five of R₆, R_{6′}, R_{6″}, R₇, R_{7′}, and R_{7″} are D.

In another aspect, the application provides a method for treating a brain tumor characterized by the presence of an IDH1 and/or IDH2 mutation in a patient in need thereof comprising administering to the patient (a) a compound of formula (I) or a pharmaceutical composition comprising a compound of formula (I)

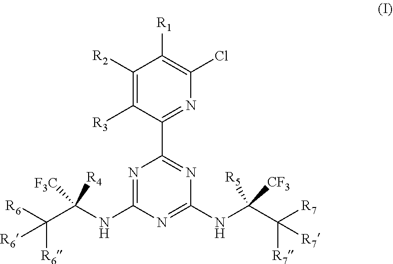

wherein each of R₁, R₂, R₃, R₄, R₅, R₆, R_{6′}, R_{6″}, R₇, R_{7′} and R_{7″} is independently H or D provided that at least one of R₁, R₂, R₃, R₄, R₅, R₆, R_{6′}, R_{6″}, R₇, R_{7′} and R_{7″} is D, or a pharmaceutically acceptable salt or a pharmaceutically acceptable co-crystalline material thereof, where D represents a deuterium atom; and (b) radiation therapy; in amounts effective for treating the brain tumor.

In one embodiment, each of R₁, R₂, R₃, R₄, R₅, R₆, R_{6′}, R_{6″}, R₇, R_{7′}, and R_{7″} is D.

In another embodiment, any one or two or all three of R₁, R₂, or R₃ is or are D and each of R₄, R₅, R₆, R_{6′}, R_{6″}, R₇, R_{7′}, and R_{7″} is H. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, one or two or all three of R₁, R₂, or R₃ is or are D and either or both of R₄ and R₅ is or are D and each of R₆, R_{6′}, R_{6″}, R₇, R_{7′}, and R_{7″} is H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and either or both of R₄ and R₅ is or are D and all three of R₆, R_{6′}, and R_{6″} are D and all three of R₇, R_{7′}, and R_{7″} are H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or R₄ is H and R₅ is D or both R₄ and R₅ are D.

In another embodiment, one or two or all three of R₁, R₂, and R₃ is or are D and both of R₄ and R₅ are H and each of R₆, R_{6′}, and R_{6″} is D and each of R₇, R_{7′}, and R_{7″} is H. In some of such embodiments, any one of R₁, R₂, or R₃ is or are D. In other such embodiments, any two of R₁, R₂, or R₃ are D. In still another of such embodiments, all three of R₁, R₂, and R₃ are D.

In another embodiment, one or two or all three of R₁, R₂, and R₃ is or are D and either or both of R₄ and R₅ is or are D and each of R₆, R_{6′} R_{6″}, R₇, R_{7′}, and R_{7″} is D. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and both of R₄ and R₅ are H and each of R₆, R_{6′}, R_{6″}, R₇, R_{7′}, and R_{7″} is D. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, all three of R₁, R₂, and R₃ are H and each of R₄, R₅, R₆, R_{6′}, R_{6″}, R₇, R_{7′}, and R_{7″} is D.

In another embodiment, all three of R₁, R₂, and R₃ are H and either of R₄ and R₅ is or are D and each of R₆, R_{6′}, R_{6″}, R₇, R_{7′}, and R_{7″} is D. In some of such embodiments, R₄ is H and R₅ is D.

In another embodiment, all three of R₁, R₂, and R₃ are H and either or both of R₄ and R₅ is or are D and all three of R₆, R_{6′}, and R_{6″} are D and all three of R₇, R_{7′}, and R_{7″} are H. In some of such embodiments, R₄ is H and R₅ is D. In other of such embodiments, R₄ is D and R₅ is H. In still other of such embodiments, both of R₄ and R₅ are D.

In another embodiment, all three of R₁, R₂, and R₃ are H and either or both of R₄ and R₅ is or are D and each of R₆, R_{6′}, R_{6″}, R₇, R_{7′}, and R_{7″} is H. In some of such embodiments, R₄ is H and R₅ is D. In still other of such embodiments, both of R₄ and R₅ are D.

In another embodiment, all three of R₁, R₂, and R₃ are H and both of R₄ and R₅ are H and each of R₆, R_{6′}, R_{6″}, R₇, R_{7′}, and R_{7″} is D.

In another embodiment, all three of R₁, R₂, and R₃ are H and both of R₄ and R₅ are H and each of R₆, R_{6′}, and R_{6″} is D and each of R₇, R_{7′}, and R_{7″} is H.

In still other embodiments of compounds of Formula I, at least one of R₆, R_{6′}, R_{6″}, R₇, R_{7′}, and R_{7″} is D. In some of such embodiments, any two or three or four or five of R₆, R_{6′}, R_{6″}, R₇, R_{7′}, and R_{7″} are D. In other such embodiments, any two of R₆, R_{6′}, R_{6″}, R₇, R_{7′}, and R_{7″} are D. In other such embodiments, any three of R₆, R_{6′}, R_{6″}, R₇, R_{7′}, and R_{7″} are D. In other such embodiments, any four of R₆, R_{6′}, R_{6″}, R₇, R_{7′}, and R_{7″} are D. In other such embodiments, any five of R₆, R_{6′}, R_{6″}, R₇, R_{7′}, and R_{7″} are D.

The radiation therapy may be administered concurrently with or sequentially with (prior to or following) the administration of the compound of formula (I). In some embodiments, the compound of formula (I) and the radiation therapy are administered concurrently. In other embodiments, the compound of formula (I) and the radiation therapy are administered sequentially.

In another aspect, the application provides a method for treating a brain tumor characterized by the presence of an IDH1 and/or IDH2 mutation in a patient in need thereof comprising administering to the patient (a) a compound of formula (I) or a pharmaceutical composition comprising a compound of formula (I)

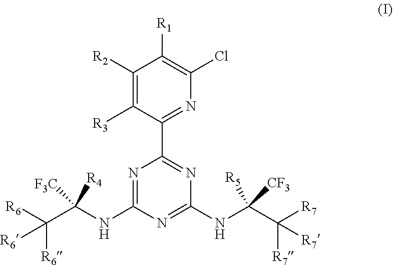

wherein each of R₁, R₂, R₃, R₄, R₅, R₆, R_{6′}, R_{6″}, R₇, R_{7′} and R_{7″} is independently H or D provided that at least one of R₁, R₂, R₃, R₄, R₅, R₆, R_{6′}, R_{6″}, R₇, R_{7′} and R_{7″} is D, or a pharmaceutically acceptable salt or a pharmaceutically acceptable co-crystalline material thereof, where D represents a deuterium atom; (b) an additional therapeutic agent and, optionally, (c) radiation therapy; in amounts effective for treating the brain tumor.

In one embodiment, each of R₁, R₂, R₃, R₄, R₅, R₆, R_{6′}, R_{6″}, R₇, R_{7′}, and R_{7″} is D.

In another embodiment, any one or two or all three of R₁, R₂, or R₃ is or are D and each of R₄, R₅, R₆, R_{6′}, R_{6″}, R₇, R_{7′}, and R_{7″} is H. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, one or two or all three of R₁, R₂, or R₃ is or are D and either or both of R₄ and R₅ is or are D and each of R₆, R_{6′}, R_{6″}, R₇, R_{7′}, and R_{7″} is H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and either or both of R₄ and R₅ is or are D and all three of R₆, R_{6′}, and R_{6″} are D and all three of R₇, R_{7′}, and R_{7″} are H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or R₄ is H and R₅ is D or both R₄ and R₅ are D.

In another embodiment, one or two or all three of R₁, R₂, and R₃ is or are D and both of R₄ and R₅ are H and each of R₆, R_{6′}, and R_{6″} is D and each of R₇, R_{7′}, and R_{7″} is H. In some of such embodiments, any one of R₁, R₂, or R₃ is or are D. In other such embodiments, any two of R₁, R₂, or R₃ are D. In still another of such embodiments, all three of R₁, R₂, and R₃ are D.

In another embodiment, one or two or all three of R₁, R₂, and R₃ is or are D and either or both of R₄ and R₅ is or are D and each of R₆, R_{6′} R_{6″}, R₇, R_{7′}, and R_{7″} is D. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and both of R₄ and R₅ are H and each of R₆, R_{6′}, R_{6″}, R₇, R_{7′}, and R_{7″} is D. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, all three of R₁, R₂, and R₃ are H and each of R₄, R₅, R₆, R_{6′}, R_{6″}, R₇, R_{7′}, and R_{7″} is D.

In another embodiment, all three of R₁, R₂, and R₃ are H and either of R₄ and R₅ is or are D and each of R₆, R_{6′}, R_{6″}, R₇, R_{7′}, and R_{7″} is D. In some of such embodiments, R₄ is H and R₅ is D.

In another embodiment, all three of R₁, R₂, and R₃ are H and either or both of R₄ and R₅ is or are D and all three of R₆, R_{6′}, and R_{6″} are D and all three of R₇, R_{7′}, and R_{7″} are H. In some of such embodiments, R₄ is H and R₅ is D. In other of such embodiments, R₄ is D and R₅ is H. In still other of such embodiments, both of R₄ and R₅ are D.

In another embodiment, all three of R₁, R₂, and R₃ are H and either or both of R₄ and R₅ is or are D and each of R₆, R_{6′}, R_{6″}, R₇, R_{7′}, and R_{7″} is H. In some of such embodiments, R₄ is H and R₅ is D. In still other of such embodiments, both of R₄ and R₅ are D.

In another embodiment, all three of R₁, R₂, and R₃ are H and both of R₄ and R₅ are H and each of R₆, R_{6′}, R_{6″}, R₇, R_{7′}, and R_{7″} is D.

In another embodiment, all three of R₁, R₂, and R₃ are H and both of R₄ and R₅ are H and each of R₆, R_{6′}, and R_{6″} is D and each of R₇, R_{7′}, and R_{7″} is H.

In still other embodiments of compounds of Formula I, at least one of R₆, R_{6′}, R_{6″}, R₇, R_{7′}, and R_{7″} is D. In some of such embodiments, any two or three or four or five of R₆, R_{6′}, R_{6″}, R₇, R_{7′}, and R_{7″} are D. In other such embodiments, any two of R₆, R_{6′}, R_{6″}, R₇, R_{7′}, and R_{7″} are D. In other such embodiments, any three of R₆, R_{6′}, R_{6″}, R₇, R_{7′}, and R_{7″} are D. In other such embodiments, any four of R₆, R_{6′}, R_{6″}, R₇, R_{7′}, and R_{7″} are D. In other such embodiments, any five of R₆, R_{6′}, R_{6″}, R₇, R_{7′}, and R_{7″} are D. In such embodiments, R₁, R₂, R₃, R₄, and R₅ may be as defined in any of the other foregoing embodiments described herein.

The additional therapeutic agent may be administered together with the compound of formula (I) in a single dosage form (e.g., pharmaceutical composition) or as a separate dosage form. If administered as a separate dosage form, the additional therapeutic agent may be administered concurrently with or sequentially with (prior to or following) the administration of the compound of formula (I). In some embodiments, the compound of formula (I) and the additional therapeutic agent are administered concurrently. In other embodiments, the compound of formula (I) and the additional therapeutic agent are administered sequentially.

As used herein, the term “cocrystal” or “co-crystalline material” refers to a crystalline solid made up of two or more neutral chemical species in a defined stoichiometric ratio that possesses distinct crystallographic and spectroscopic properties when compared to the species individually. A “cocrystal” is distinct from a “salt,” which is made up of charged-balanced charged species. The species making up a cocrystal typically are linked by hydrogen bonding and other non-covalent and non-ionic interactions.

Thus, a pharmaceutical cocrystal of a drug typically comprises the drug and one or more coformers. The combinations of drug and coformer(s) that will form cocrystals generally cannot be predicted ab initio, and cocrystal formation typically affects the physicochemical properties of a drug in unpredictable ways.

As used herein, the term “crystalline” refers to a solid material whose constituent particles (e.g., molecules) are arranged spatially in a regular and repeating lattice.

As used herein, the phrase “amounts effective” refers to the amounts of a compound of formula (I) and radiation therapy and/or an additional therapeutic agent that are sufficient to achieve a therapeutic benefit for treating a brain tumor in the methods described herein. The amounts effective in the methods described herein may or may not be the same as the amounts that are effective when any of the compounds of formula (I), radiation therapy, or additional therapeutic agent is administered as a monotherapy. In some embodiments, the amount of a compound of formula (I) that is effective in the methods described herein is the same as, less than, or more than the amount of the compound of formula (I) that is effective when the compound of formula (I) is administered as a monotherapy. In some embodiments, the amount of radiation therapy that is effective in the methods described herein is the same as, less than, or more than the amount of radiation therapy that is effective when the radiation therapy is administered as a monotherapy. In some embodiments, the amount of the additional therapeutic agent that is effective in the methods described herein is the same as, less than, or more than the amount of the additional therapeutic agent that is effective when the additional therapeutic agent is administered as a monotherapy.

As used herein, the term “treating,” when referring to a brain tumor, means having a therapeutic effect on, alleviating one or more symptoms of, altering the progression of, eradicating, reducing the size of, slowing or inhibiting the growth of, delaying or minimizing one or more symptoms associated with, reducing the malignancy of, and/or inducing stasis of the brain tumor. In some embodiments, “treating” comprises reducing the size of and/or slowing or inhibiting the growth of the brain tumor. In some embodiments, “treating” comprises reducing the size of and/or slowing or inhibiting the growth of the brain tumor for a period of time, followed by stasis of the brain tumor. In some embodiments, “treating” comprises having a therapeutic effect on, alleviating the symptoms of, altering the progression of, and/or inducing stasis of the brain tumor without affecting the size of the brain tumor. In some embodiments, “treating” comprises reducing the number or percentage of malignant cells in a brain tumor.

In one embodiment, the methods provided herein provide a complete response, partial response or stable disease in patients having a brain tumor characterized by the presence of an IDH1 and/or IDH2 mutation.

In one embodiment, the methods provided herein increase the overall survival of patients having a brain tumor characterized by the presence of an IDH1 and/or IDH2 mutation when treated with an effective amount of a compound of formula (I) as compared to patients that are not treated with a compound of formula (I).

In one embodiment, the methods provided herein increase the complete remission rate of patients having a brain tumor characterized by the presence of an IDH1 and/or IDH2 mutation when treated with an effective amount of a compound of formula (I) as compared to patients that are not treated with a compound of formula (I).

In one embodiment, the methods provided herein increase the objective response rate of patients having a brain tumor characterized by the presence of an IDH1 and/or IDH2 mutation when treated with an effective amount of a compound of formula (I) as compared to patients that are not treated with a compound of formula (I).

In one embodiment, the methods provided herein increase the time to progression of patients having a brain tumor characterized by the presence of an IDH1 and/or IDH2 mutation when treated with an effective amount of a compound of formula (I) as compared to patients that are not treated with a compound of formula (I).

In one embodiment, the methods provided herein increase the relapse free survival of patients having a brain tumor characterized by the presence of an IDH1 and/or IDH2 mutation when treated with an effective amount of a compound of formula (I) as compared to patients that are not treated with a compound of formula (I).

In one embodiment, the methods provided herein increase the progression free survival of patients having a brain tumor characterized by the presence of an IDH1 and/or IDH2 mutation when treated with an effective amount of a compound of formula (I) as compared to patients that are not treated with a compound of formula (I).

In one embodiment, the methods provided herein increase the event-free survival of patients having a brain tumor characterized by the presence of an IDH1 and/or IDH2 mutation when treated with an effective amount of a compound of formula (I) as compared to patients that are not treated with a compound of formula (I).

In one embodiment, the methods provided herein increase the duration of remission of patients having a brain tumor characterized by the presence of an IDH1 and/or IDH2 mutation when treated with an effective amount of a compound of formula (I) as compared to patients that are not treated with a compound of formula (I).

In one embodiment, the methods provided herein increase the duration or response of patients having a brain tumor characterized by the presence of an IDH1 and/or IDH2 mutation when treated with an effective amount of a compound of formula (I) as compared to patients that are not treated with a compound of formula (I).

In one embodiment, the methods provided herein decrease the time to remission/response of patients having a brain tumor characterized by the presence of an IDH1 and/or IDH2 mutation when treated with an effective amount of a compound of formula (I) as compared to patients that are not treated with a compound of formula (I).

As used herein, the term “complete response” (CR) refers to the disappearance of all signs of cancer in response to treatment or that one or more manifestations of disease are below the limits of detection of a particular analytical method, such as, for example, by MRI (magnetic resonance imaging). This does not always mean the cancer has been cured. The term is also interchangeable in the art with “complete remission.”

As used herein, the term “partial response” refers to a decrease in the size of a tumor, or in the extent of cancer in the body, in response to treatment. The term is also interchangeable in the art with “partial remission.”

As used herein, the term “stable disease” refers to cancer that is neither increasing nor decreasing in extent or severity.

As used herein, the term “overall survival” (OS) means the time from randomization in a clinical trial until death from any cause.

As used herein, the term “complete remission rate” refers to complete disappearance of all such manifestations of disease or that one or more manifestations of disease are below the limits of detection of a particular analytical method, such as, for example, by MRI (magnetic resonance imaging).

As used herein, the term “objective response rate” (ORR) refers to the proportion of patients with tumor size reduction of a predefined amount and for a minimum time period. Response duration usually is measured from the time of initial response until documented tumor progression. Generally, the U.S. FDA has defined ORR as the sum of partial responses plus complete responses. When defined in this manner, ORR is a direct measure of drug antitumor activity, which can be evaluated in a single-arm study. Stable disease should not be a component of ORR. Stable disease can reflect the natural history of disease, whereas tumor reduction is a direct therapeutic effect. The significance of ORR is assessed by its magnitude and duration, and the percentage of complete responses (no detectable evidence of tumor).

As used herein, the term “time to progression” (TPP) refers to the time from randomization until objective tumor progression; TTP does not include deaths.

As used herein, the term “relapse-free survival” (RFS) refers to the length of time after primary treatment for a cancer ends that the patient survives without any signs or symptoms of that cancer. In a clinical trial, measuring the relapse-free survival is one way to see how well a new treatment works. The term is also interchangeable in the art as disease-free survival (DFS).

As used herein, the term “progression-free survival” (PFS) means the time from randomization in a clinical trial until progression or death.

As used herein, the term “event-free survival” (EFS) means the time from study entry until any treatment failure, including disease progression, treatment discontinuation for any reason, or death.

As used herein, the term “duration of response” (DoR) is the time from achieving a response until relapse or disease progression.

As used herein, the term “patient” refers to a mammal, including mice, rats, dogs and humans, which is afflicted with a brain tumor (e.g., a glioma). In some embodiments, the patient is a human. In some embodiments the patient is a human child. In other embodiments the patient is an adolescent.

In some embodiments, a compound of formula (I) is administered in an amount of from 1 to 250 mg/day, 5 to 100 mg/day, 8 to 75 mg/day, 10 to 50 mg/day, 15 to 40 mg/day, or 20 to 30 mg/day. In other embodiments, a compound of formula (I) is administered in an amount of from 0.01 to 10 mg/kg of body weight per day, 0.2 to 8.0 mg/kg of body weight per day, 0.4 to 6.0 mg/kg of body weight per day, 0.6 to 4.0 mg/kg of body weight per day, 0.8 to 2.0 mg/kg of body weight per day, 0.1 to 1 mg/kg of body weight per day, 0.2 to 1.0 mg/kg of body weight per day, 0.15 to 1.5 mg/kg of body weight per day, or 0.1 to 0.5 mg/kg of body weight per day. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and/or the judgment of the treating physician.

In some embodiments, the radiation therapy is administered in a manner consistent with the National Comprehensive Cancer Network Clinical Practice Guidelines in Oncology (e.g., dose and schedule of administration), version 1.2016 available at nccn.org.

In some embodiments, the radiation therapy is administered in a cumulative dose of 20-100 Gy, or 30-80 Gy, or 30-60 Gy, or 40-70 Gy, or 40-60 Gy, or 30-40 Gy, or 40-50 Gy, or 50-60 Gy, or 45-55 Gy, in 1.0-5.0 Gy fractions, or 1.5-3.0 Gy fractions, or 1.0-1.5 Gy fractions, or 1.5-2.0 Gy fractions, or 2.0-2.5 Gy fractions, or 2.5-3.0 Gy fractions, or 1.8-2.0 Gy fractions, or 1.8 Gy fractions, or 2.0 Gy fractions. In some embodiments, the radiation therapy is administered in a cumulative dose of 50-70 Gy in 1.5-2.5 Gy fractions, or 60 Gy in 2.0 Gy fractions. The cumulative dose refers to the total of all of the fractional doses given during a course of treatment.

The dose of radiation therapy may be selected based on the nature of the brain tumor. In some embodiments where the brain tumor is a low grade glioma, the radiation therapy is administered in a cumulative dose of 40-50 Gy in 1.5-2.5 Gy fractions, or in a cumulative dose of 45-54 Gy in 1.8-2.0 Gy fractions, or in a cumulative dose of 45.5 Gy in 1.8-2.0 Gy fractions. In some embodiments where the brain tumor is a high grade glioma, the radiation therapy is administered in a cumulative dose of 50-70 Gy in 1.5-2.5 Gy fractions, or in a cumulative dose of 59.4 Gy in 1.8 Gy fractions, or in a cumulative dose of 55.8-59.4 Gy in 1.8 Gy fractions, or in a cumulative dose of 57 Gy in 1.9 Gy fractions, or in a cumulative dose of 60 Gy in 1.8-2.0 Gy fractions, or 25 Gy in 5.0 Gy fractions. In some embodiments where the brain tumor is a glioblastoma, the radiation therapy is administered in a cumulative dose of 30-60 Gy in 2.0-4.0 Gy fractions, or in a cumulative dose of 34 Gy in 3.4 Gy fractions, or in a cumulative dose of 35-45 Gy in 2.5-3.0 Gy fractions, or in a cumulative dose of 50 Gy in 2.5 Gy fractions.

Additional Therapeutic Agents

As used here, the “one or more additional therapeutic agents” employed in the methods described herein include those agents that are known to be useful for treating brain tumors, i.e., having a therapeutic effect on, alleviating one or more symptoms of, altering the progression of, eradicating, reducing the size of, slowing or inhibiting the growth of, delaying or minimizing one or more symptoms associated with, reducing the malignancy of, or inducing stasis of the brain tumor, or alleviating or minimizing one or more side effects associated with another therapy applied or administered to treat the brain tumor.

In some embodiments, the one or more additional therapeutic agents include one or more of a DNA-reactive agent, a PARP inhibitor, an anti-emesis agent, an anti-convulsant or anti-epileptic agent, a checkpoint inhibitor, PVC chemotherapy, bevacizumab, and/or gemcitabine.

An example of an “additional therapeutic agent” is a DNA-reactive agent. As used herein, “DNA-reactive agents” are those agents, such as alkylating agents, cross-linking agents, and DNA intercalating agents, which interact covalently or non-covalently with cellular DNA. For example, DNA-reactive agents include adozelesin, altretamine, bizelesin, busulfan, carboplatin, carboquone, carmustine, chlorambucil, cisplatin, cyclophosphamide, dacarbazine, estramustine, fotemustine, hepsulfam, ifosfamide, improsulfan, irofulven, lomustine, mechlorethamine, melphalan, mitozolomide, nedaplatin, oxaliplatin, piposulfan, procarbazine, semustine, streptozocin, temozolomide, thiotepa, treosulfan, diethylnitrosoamine, benzo(a)pyrene, doxorubicin, mitomycin-C, and the like.

Many of these DNA-reactive agents are useful in cancer therapy as DNA-reactive chemotherapeutic agents.

In some embodiments, the DNA-reactive agent is temozolomide (TMZ). In one aspect of these embodiments, the TMZ is administered in a manner consistent with the National Comprehensive Cancer Network Clinical Practice Guidelines in Oncology (e.g., dose and schedule of administration), version 1.2016 available at nccn.org. In one aspect of these embodiments, the TMZ is administered in a manner consistent with the prescribing information for TEMODAR® (temozolomide) Capsules and TEMODAR® (temozolomide) for Injection. In some aspects of these embodiments, the TMZ is administered in a daily dose of 100-250 mg/m² based on the patient's body surface area, or 100-150 mg/m², or 150-200 mg/m², or 200-250 mg/m². In some aspects of these embodiments, the TMZ is administered in a daily dose of 50-100 mg/m² based on the patient's body surface area, or 50-75 mg/m², or 75-100 mg/m², or 60-90 mg/m², or 65-85 mg/m², or 70-80 mg/m². In some aspects of these embodiments, the TMZ is administered in a daily dose of 125-175 mg/m² based on the patient's body surface area for 5 consecutive days of a 28-day treatment cycle. In some aspects of these embodiments, the TMZ is administered in combination with radiation therapy in a daily dose of 50-100 mg/m² based on the patient's body surface area, or 50-75 mg/m², or 75-100 mg/m², or 60-90 mg/m², or 65-85 mg/m², or 70-80 mg/m².

In some aspects of these embodiments, the TMZ is administered in combination with radiation therapy in a daily dose of 70-80 mg/m² based on the patient's body surface area for 42 days. In some aspects of these embodiments where the brain tumor is a high-grade glioma or glioblastoma, the TMZ is administered in combination with radiation therapy in a daily dose of 70-80 mg/m² based on the patient's body surface area for 42 days. In some aspects of these embodiments where the brain tumor is an anaplastic astrocytoma, the TMZ is administered in a daily dose of 125-175 mg/m² based on the patient's body surface area for 5 consecutive days of a 28-day treatment cycle. In some aspects of these embodiments where the brain tumor is an anaplastic astrocytoma, the TMZ is administered in a daily dose of 175-225 mg/m² based on the patient's body surface area for 5 consecutive days of a 28-day treatment cycle.

In some embodiments, the one or more additional therapeutic agents is a PARP inhibitor. As used herein, “PARP inhibitor” refers to an inhibitor of the enzyme poly ADP ribose polymerase (PARP). Examples of PARP inhibitors include pamiparib, olaparib, rucaparib, velaparib, iniparib, talazoparib, niraparib, and the like.

In some embodiments, the one or more additional therapeutic agents is an anti-emesis agent. As used herein, “anti-emesis agent” or “antiemetic” refers to a drug that is effective to reduce vomiting and nausea symptoms. Examples of anti-emesis agents include 5-HT3 receptor antagonists (e.g., dolasetron, granisetron, ondansetron, tropisetron, palonosetron, mirtazapine, and the like), dopamine agonists (e.g., domperidone, olanzapine, droperidol, haloperidol, chlorpromazine, prochlorperazine, alizapride, prochlorperazine, metoclopramide, and the like), NK1 receptor antagonists (e.g., aprepitant, casopitant, rolapitant, and the like), antihistamines (e.g., cinnarizine, cyclizine, diphenhydramine, dimenhydrinate, doxylamine, meclizine, promethazine, hydroxyzine, and the like), cannabinoids (e.g, cannabis, dronabinol, synthetic cannabinoids, and the like), benzodiazepines (e.g., midazolam, lorazepam, and the like), anticholinergics (e.g., scopolamine and the like), steroids (e.g, dexamethasone and the like), trimethobenzamide, ginger, propofol, glucose/fructose/phosphoric acid (which is sold under the trade name Emetrol®), peppermint, muscimol, ajwain, and the like.

In some embodiments, the one or more additional therapeutic agents is an anti-convulsant or anti-epileptic agent. As used herein, “anti-convulsant or anti-epileptic agent” refers to a drug that is effective for treating or preventing seizures, including epileptic seizures. Examples of anti-convulsants include paraldehyde, stiripentol, phenobarbital, methylphenobarbital, barbexaclone, clobazam, clonazepam, clorazepate, diazepam, midazolam, lorazepam, nitrazepam, temazepam, nimetazepam, potassium bromide, felbamate, carbamazepine, oxcarbazepine, eslicarbazepine acetate, valproic acid, sodium valproate, divalproex sodium, vigabatrin, progabide, tiagabine, topiramate, gabapentin, pregabalin, ethotoin, phenytoin, mephenytoin, fosphenytoin, paramethadione, trimethadione, ethadione, beclamide, primidone, brivaracetam, etiracetam, levetiracetam, seletracetam, ethosuximide, phensuximide, mesuximide, acetazolamide, sultiame, methazolamide, zonisamide, lamotrigine, pheneturide, phenacemide, valpromide, valnoctamide, perampanel, stiripentol, pyridoxine, and the like.

In some embodiments, the one or more additional therapeutic agents is a checkpoint inhibitor. As used herein, “checkpoint inhibitor” refers to a therapeutic agent that inhibits an immune checkpoint (e.g., CTLA-4, PD-1/PD-L1, and the like) that otherwise would prevent immune system attacks on cancer cells, thereby allowing the immune system to attack the cancer cells. Examples of check point inhibitors include ipilimumab, nivolumab, pembrolizumab, atezolizumab, avelumab, durvalumab, BGB-A317, spartalizumab, and the like.

In some embodiments, the one or more additional therapeutic agents is PVC chemotherapy. As used herein, “PVC chemotherapy” refers to a chemotherapy regimen comprising the combined administration of procarbazine, lomustine (which is sold under the trade name CCNU®), and vincristine (which is sold under the trade name Onocovin®). Typically, the vincristine is administered intravenously, while the procarbazine, and lomustine are administered orally. PCV chemotherapy often is administered in cycles, wherein each cycle comprises a single administration of vincristine and lomustine and a 10-day course of treatment with procarbazine.

In some embodiments, the one or more additional therapeutic agents is bevacizumab. Bevacizumab, which is sold under the trade name Avastin®, is a recombinant humanized monoclonal antibody.

In some embodiments, the one or more additional therapeutic agents is gemcitabine. Gemcitabine, which is sold under the trade name Gemzar®, is a pyrimidine nucleoside analog.

Deuterated Compounds

The compounds of this application contain one or more asymmetric centers and thus may exist as racemates, racemic mixtures, scalemic mixtures, and/or diastereomeric mixtures, as well as single enantiomers or individual stereoisomers that are substantially free from another possible enantiomer or stereoisomer. The term “substantially free of other stereoisomers” as used herein means a preparation enriched in a compound having a selected stereochemistry at one or more selected stereocenters by at least about 60%, 65%, 70%, 75%, 80%, 85%, 90%, 95%, 96%, 97%, 98%, or 99%. The term “enriched” means that at least the designated percentage of a preparation is the compound having a selected stereochemistry at one or more selected stereocenters. Methods of obtaining or synthesizing an individual enantiomer or stereoisomer for a given compound are known in the art and may be applied as practicable to final compounds or to starting material or intermediates.

In one embodiment, the compound is enriched in a specific stereoisomer by at least about 60%, 65%, 70%, 75%, 80%, 85%, 90%, 95%, 96%, 97%, 98%, or 99%.

As used herein, the term “isotopic enrichment factor” refers to the ratio between the isotopic abundance of a given isotope at a designated position of a compound and the natural abundance of that isotope. The skilled artisan would understand how to prepare compounds with varying degrees of isotopic enrichment at a particular hydrogen atom. It should be understood that the natural abundance of deuterium has no bearing on the designation of any site as having an “H” or “hydrogen” present. Therefore, any level of desired enrichment can be prepared by the skilled artisan. In some embodiments, the positions designated specifically as “D” or “deuterium” in the compound of the application shall be understood to have an isotopic enrichment factor for each designated deuterium atom of at least 6000 (90% deuterium incorporation), at least 6333 (95% deuterium incorporation), or at least 6600 (99% deuterium incorporation. In some embodiments, the compounds described herein shall be understood to have an isotopic enrichment factor of at least 6600 (99% deuterium incorporation) or greater at positions that are specifically designated as “D” or “deuterium”.

Compounds described herein may be prepared following procedures detailed in the examples and other analogous methods known to one skilled in the art. Compounds produced by any of the schemes set forth below may be further modified (e.g., through the addition of substituents to rings, etc.) to produce additional compounds. For example, compounds bearing deuterium at positions other than the positions shown in the examples can be prepared by similar methods using suitable starting materials. The specific approaches and compounds shown herein are not intended to be limiting. The suitability of a particular chemical group (as well as alternate chemical groups) in a compound structure for use in the synthesis of another compound is within the knowledge of one of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene, T W et al., Protective Groups in Organic Synthesis, 3^{rd} Ed., John Wiley and Sons (1999); Fieser, L et al., Fieser and Fieser's Reagents for Organic Synthesis, John Wiley and Sons (1994); and Paquette, L, ed., Encyclopedia of Reagents for Organic Synthesis, John Wiley and Sons (1995) and subsequent editions thereof.

The starting materials and/or intermediate compounds are known in the art or can be prepared in accordance with methods known to those skilled in the art as exemplified by the methods described in US2015/0018328 A1CN102875270(B), DE3900300(A1), Braid. M. et al., J. Amer. Chem. Soc. 76: 4027 (1954), Ishii, A. et al., Synlett 12:1381 (1997), and Packer, G. et al., Tetrahedron Asymmetry 28:539 (2017).

It may be convenient or desirable to prepare, purify, and/or handle a corresponding salt of the active compound, for example, a pharmaceutically acceptable salt. Examples of pharmaceutically acceptable salts are discussed in Berge et al., 1977, “Pharmaceutically Acceptable Salts.” J. Pharm. Sci. Vol. 66, pp. 1-19.

If the compound is cationic, or has a functional group that may be cationic (e.g., —NH₂ may be —NH₃*), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms and co-crystalline materials thereof.

In one aspect, the disclosure relates to a cocrystal comprising a compound of formula (I)

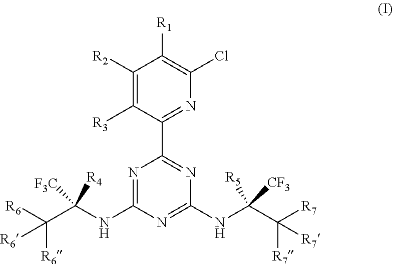

and citric acid (hereinafter “citric acid cocrystal”), wherein each of R₁, R₂, R₃, R₄, R₅, R₆, R_{6′}, R_{6″}, R₇, R_{7′} and R_{7″} is independently H or D provided that at least one of R₁, R₂, R₃, R₄, R₅, R₆, R_{6′}, R_{6″}, R₇, R_{7′} and R_{7″} is D, where D represents a deuterium atom.

In one embodiment, each of R₁, R₂, R₃, R₄, R₅, R₆, R_{6′}, R_{6″}, R₇, R_{7′}, and R_{7″} is D.

In another embodiment, any one or two or all three of R₁, R₂, or R₃ is or are D and each of R₄, R₅, R₆, R_{6′}, R_{6″}, R₇, R_{7′}, and R_{7″} is H. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, one or two or all three of R₁, R₂, or R₃ is or are D and either or both of R₄ and R₅ is or are D and each of R₆, R_{6′}, R_{6″}, R₇, R_{7′}, and R_{7″} is H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and either or both of R₄ and R₅ is or are D and all three of R₆, R_{6′}, and R_{6″} are D and all three of R₇, R_{7′}, and R_{7″} are H. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or R₄ is H and R₅ is D or both R₄ and R₅ are D.

In another embodiment, one or two or all three of R₁, R₂, and R₃ is or are D and both of R₄ and R₅ are H and each of R₆, R_{6′}, and R_{6″} is D and each of R₇, R_{7′}, and R_{7″} is H. In some of such embodiments, any one of R₁, R₂, or R₃ is or are D. In other such embodiments, any two of R₁, R₂, or R₃ are D. In still another of such embodiments, all three of R₁, R₂, and R₃ are D.

In another embodiment, one or two or all three of R₁, R₂, and R₃ is or are D and either or both of R₄ and R₅ is or are D and each of R₆, R_{6′} R_{6″}, R₇, R_{7′}, and R_{7″} is D. In some of such embodiments, any one of R₁, R₂, or R₃ is D and either or both of R₄ and R₅ is or are D. In other such embodiments, any two of R₁, R₂, or R₃ are D and either or both of R₄ and R₅ is or are D. In still another of such embodiments, all three of R₁, R₂, and R₃ are D and either or both of R₄ and R₅ is or are D. In any of the foregoing embodiments, R₄ is D and R₅ is H or both R₄ and R₅ are D.

In another embodiment, one or two or all three of R₁, R₂, and R₃ is or are D and both of R₄ and R₅ are H and each of R₆, R_{6′}, R_{6″}, R₇, R_{7′}, and R_{7″} is D. In some of such embodiments, any one of R₁, R₂, or R₃ is D. In other such embodiments, any two of R₁, R₂, or R₃ are D. In still another of such embodiments, all three of R₁, R₂, and R₃ are D.

In another embodiment, all three of R₁, R₂, and R₃ are H and each of R₄, R₅, R₆, R_{6′}, R_{6″}, R₇, R_{7′}, and R_{7″} is D.

In another embodiment, all three of R₁, R₂, and R₃ are H and either of R₄ and R₅ is or are D and each of R₆, R_{6′}, R_{6″}, R₇, R_{7′}, and R_{7″} is D. In some of such embodiments, R₄ is H and R₅ is D.

In another embodiment, all three of R₁, R₂, and R₃ are H and either or both of R₄ and R₅ is or are D and all three of R₆, R_{6′}, and R_{6″} are D and all three of R₇, R_{7′}, and R_{7″} are H. In some of such embodiments, R₄ is H and R₅ is D. In other of such embodiments, R₄ is D and R₅ is H. In still other of such embodiments, both of R₄ and R₅ are D.

In another embodiment, all three of R₁, R₂, and R₃ are H and either or both of R₄ and R₅ is or are D and each of R₆, R_{6′}, R_{6″}, R₇, R_{7′}, and R_{7″} is H. In some of such embodiments, R₄ is H and R₅ is D. In still other of such embodiments, both of R₄ and R₅ are D.

In another embodiment, all three of R₁, R₂, and R₃ are H and both of R₄ and R₅ are H and each of R₆, R_{6′}, R_{6″}, R₇, R_{7′}, and R_{7″} is D.

In another embodiment, all three of R₁, R₂, and R₃ are H and both of R₄ and R₅ are H and each of R₆, R_{6′}, and R_{6″} is D and each of R₇, R_{7′}, and R_{7″} is H.

In still other embodiments of compounds of Formula I, at least one of R₆, R_{6′}, R_{6″}, R₇, R_{7′}, and R_{7″} is D. In some of such embodiments, any two or three or four or five of R₆, R_{6′}, R_{6″}, R₇, R_{7′}, and R_{7″} are D. In other such embodiments, any two of R₆, R_{6′}, R_{6″}, R₇, R_{7′}, and R_{7″} are D. In other such embodiments, any three of R₆, R_{6′}, R_{6″}, R₇, R_{7′}, and R_{7″} are D. In other such embodiments, any four of R₆, R_{6′}, R_{6″}, R₇, R_{7′}, and R_{7″} are D. In other such embodiments, any five of R₆, R_{6′}, R_{6″}, R₇, R_{7′}, and R_{7″} are D.

The citric acid cocrystals of compounds of formula (I) disclosed herein can be prepared by a person of skill in the art via methods analogous to those disclosed in WIPO Publication No. 2019/090059 A1, which is incorporated herein by reference. The publication describes pharmaceutically acceptable salts and pharmaceutically acceptable co-crystalline materials of Compound X.

In some embodiments, the citric acid cocrystal further comprises water.

In some embodiments, the citric acid cocrystal comprises a compound of formula (I), citric acid, and water in a molar ratio of 2:1:1. As a person of ordinary skill would understand, the measured molar ratio of the compound of formula (I), citric acid, and water in a given sample of the cocrystal may differ slightly from 2:1:1 due to the experimental error associated with available analytical methods, the presence of impurities (e.g., water or citric acid that is not incorporated in the crystal lattice), etc. It will be understood that cocrystals having a molar ratio of 2:1:1 fall within this embodiment, even if the measured ratio of the compound of formula (I), citric acid, and water differs slightly from 2:1:1.

In some embodiments, the citric acid cocrystal comprises four molecules of a compound of formula (I), two citric acid molecules, and two water molecules per unit cell.

As used herein, the term “unit cell’ refers to the smallest group of particles (e.g., molecules) in a crystalline solid that makes up the repeating pattern of the crystalline solid. In a cocrystal, the term “unit cell” refers to the smallest group of the two or more neutral chemical species that makes up the repeating pattern of the cocrystal.

In one aspect, the disclosure relates to a method of preparing a citric acid cocrystal, comprising

dissolving a compound of formula (I) and citric acid in a solvent to afford a solution; and

precipitating the cocrystal.

The citric acid employed in the method may be crystalline or amorphous and may be in any state of hydration or solvation. In some embodiments, the citric acid is anhydrous citric acid or citric acid monohydrate. In other embodiments, the citric acid is anhydrous citric acid. In other embodiments, the citric acid is citric acid monohydrate.

The solvent employed in the method may be any liquid or mixture of liquids suitable to dissolve the compound of formula (I) and citric acid. In some embodiments, the solvent comprises a polar organic solvent, such as methanol, ethyl acetate, acetonitrile, acetone, THF (e.g., THF/water (9:1 v/v)), or n-butanol (e.g., n-butanol/heptanes (1/3 v/v)). In some embodiments, the solvent comprises acetonitrile or acetone.

The compound of formula (I) and citric acid may be dissolved in the solvent in any molar ratio and in any concentration that allows for subsequent precipitation of the cocrystal from the solution. In some embodiments, the compound of formula (I) and citric acid are contacted with the solvent in a molar ratio of between about 1:2 and 4:1, or a molar ratio between about 1:1 and 3:1, or a molar ratio between about 1.5:1 and 2.5:1, or a molar ratio of about 2:1. In some embodiments, the amount of the compound of formula (I) contacted with the solvent is sufficient to form about a 0.01 M to 3 M solution, or about a 1 M to 2 M solution, or about a 1.5 M solution, based on the amount of the compound of formula (I). As a person of ordinary skill in the art would understand, however, in the event that some of the compound of formula (I) and/or citric acid does not dissolve in the solvent, the actual molar ratio of citric acid and the compound of formula (I) in solution, and the actual concentration of the compound of formula (I) the solution, may differ from that which would be calculated from the amounts of the compound of formula (I) and citric acid contacted with the solvent.

Synthesis

Preparation of Starting Materials and Intermediates

Undeuterated, partially deuterated, and fully deuterated methyl 6-chloropicolinate and undeuterated and partially and fully deuterated (R)-1,1,1-trifluoropropan-2-amine hydrochloride are either commercially available or can be prepared from commercially available starting materials using procedures described here or known to those skilled in the art.

### Preparation of (R)-1,1,1-trifluoropropan-2-amine (Undeuterated)

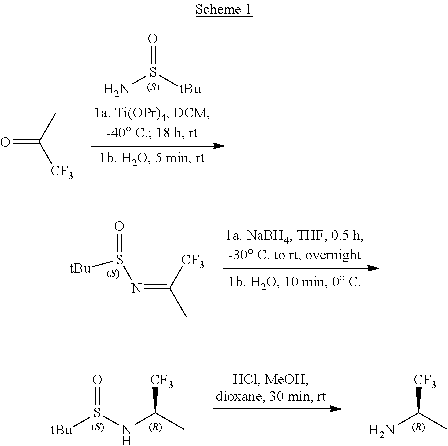

Step A: Undeuterated (RS,R)-2-methyl-N-(1,1,1-trifluoropropan-2-yl)propane-2-sulfinamide. Trifluoroacetone is commercially available. Trifluoroacetone may be converted to (R)-1,1,1-trifluoropropan-2-amine in accordance with the method described in Packer, G. et al. Tetrahedron: Asymmetry, 28:539-544 (2017).

Trifluoroacetone (5 mL, 55.8 mmol, 1 equiv) is cooled down to −40° C. in a card ice/acetone bath and then added via syringe to a sealed flask fitted with an argon balloon and containing dry hexane (50 mL), previously placed in an ice bath. (R)-(+)-2-Methylpropane-2-sulfinamide (R)-4 (dried under vacuum beforehand, 10.1 g, 121.2 mmol, 1.5 equiv) is then added at 0° C., and the flask is rinsed with dry CH₂Cl₂ (50 mL). Titanium isopropoxide (25 mL, 83.7 mmol, 1.5 equiv) is added at 0° C., and the flask is fitted with its corresponding screw top lid. The resulting mixture is allowed to warm up to r.t. (room temperature) and stirred for 48 h. The reaction mixture is then cooled down to −30° C., and flushed with an argon balloon, after which THF (70 mL) is added, followed by NaBH₄ (6.2 g, 164.5 mmol, 3.0 equiv). The mixture is stirred at −30° C. for 0.5 h, and then allowed to warm up to r.t. and stirred overnight. The reaction is cooled to 0° C. with vigorous stirring. Water (60 mL) is then added dropwise and the reaction stirred for 10 min. The reaction mixture is filtered through Celite and the filter cake is washed with CH₂Cl₂. The resulting mixture is evaporated under reduced pressure to give a solid. The latter is partitioned between CH₂Cl₂ (100 mL) and a saturated solution of NH₄Cl (60 mL). Phases are separated, and 10 mL of HCl (2 M) is added to the aqueous phase, which is back extracted with CH₂Cl₂ (3×50 mL). Brine is eventually added during the extraction process to facilitate decantation. Organic phases are combined, dried over MgSO₄ and concentrated to give the crude trifluorosulfinamide as a mixture of diastereoisomers (dr 98:2). Column chromatography (petroleum ether/EtOAc 85:15 to 6:4) affords the expected product (RS,R)-2-methyl-N-(1,1,1-trifluoropropan-2-yl)propane-2-sulfinamide as an oil (6.3 g, 52%). The column is then eluted with acetone to recover the excess (R)-(+)-2-methylpropane-2-sulfinamide.

Step B: Undeuterated (R)-1,1,1-trifluoropropan-2-amine. To a stirred solution of undeuterated RS, R)-2-methyl-N-(1,1,1-trifluoropropan-2-yl)propane-2-sulfinamide (0.56 g, 2.58 mmol, 96:4 d.r.) in MeOH (3.4 mL) is added dropwise a 4 M HCl in dioxane solution (2.60 mL, 10.31 mmol) and the reaction stirred at r.t for 30 min. Upon completion the reaction is concentrated in vacuo to yield a residue. Et2O (15 mL) is added to the resultant solid/reside in order to precipitate undeuterated (R)-1,1,1-trifluoropropan-2-amine HCl, which is filtered and washed with Et2O (2×5 mL) to yield undeuterated (R)-1,1,1-trifluoropropan-2-amine HCl as a solid with an expected 92% ee.

### Preparation of (R)-1,1,1-trifluoropropan-2-d-2-amine (Partially Deuterated)

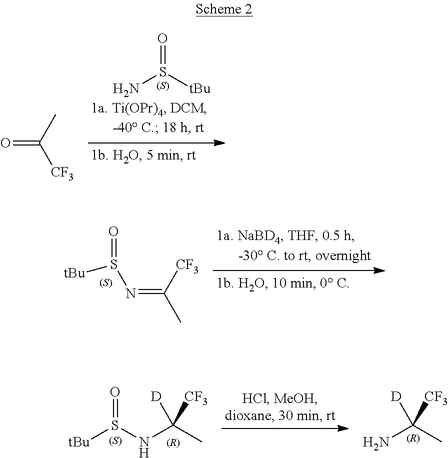

Step A: 2-Methyl-N—((R)-1,1,1-trifluoropropan-2-yl-2-d)propane-2-sulfinamide. Trifluoroacetone is commercially available. Trifluoroacetone may be converted to (R)-1,1,1-trifluoropropan-2-amine in accordance with the method described in Packer, G. et al. Tetrahedron: Asymmetry, 28:539-544 (2017).

Trifluoroacetone (5 mL, 55.8 mmol, 1 equiv) is cooled down to −40° C. in a card ice/acetone bath and then added via syringe to a sealed flask fitted with an argon balloon and containing dry hexane (50 mL), previously placed in an ice bath. (R)-(+)-2-Methylpropane-2-sulfinamide (R)-4 (dried under vacuum beforehand, 10.1 g, 121.2 mmol, 1.5 equiv) is then added at 0° C., and the flask is rinsed with dry CH₂Cl₂ (50 mL). Titanium isopropoxide (25 mL, 83.7 mmol, 1.5 equiv) is added at 0° C., and the flask is fitted with its corresponding screw top lid. The resulting mixture is allowed to warm up to r.t. and stirred for 48 h. The reaction mixture is then cooled down to −30° C., and flushed with an argon balloon, after which THF (70 mL) is added, followed by NaBD₄ (6.2 g, 164.5 mmol, 3.0 equiv). The mixture is stirred at −30° C. for 0.5 h, and then allowed to warm up to r.t. and stirred overnight. The reaction is cooled to 0° C. with vigorous stirring. Water (60 mL) is then added dropwise and the reaction stirred for 10 min. The reaction mixture is filtered through Celite and the filter cake is washed with CH₂Cl₂. The resulting mixture is evaporated under reduced pressure to give a solid. The latter is partitioned between CH₂Cl₂ (100 mL) and a saturated solution of NH₄Cl (60 mL). Phases are separated, and 10 mL of HCl (2 M) is added to the aqueous phase, which is back extracted with CH₂Cl₂ (3×50 mL). Brine is eventually added during the extraction process to facilitate decantation. Organic phases are combined, dried over MgSO₄ and concentrated to give the crude trifluorosulfinamide as a mixture of diastereoisomers (dr 98:2). Column chromatography (petroleum ether/EtOAc 85:15 to 6:4) affords the expected product (RS,R)-2-Methyl-N-(1,1,1-trifluoropropan-2-yl-2-d)propane-2-sulfinamide as an oil (6.3 g, 52%). The column is then eluted with acetone to recover the excess (R)-(+)-2-methylpropane-2-sulfinamide.

Step B: (R)-1,1,1-trifluoropropan-2-d-2-amine. To a stirred solution of (RS,R)-2-Methyl-N-(1,1,1-trifluoropropan-2-yl-2-d)propane-2-sulfinamide (2.58 mmol, 96:4 d.r.) in MeOH (3.4 mL) is added dropwise a 4 M HCl in dioxane solution (2.60 mL, 10.31 mmol) and the reaction stirred at r.t. for 30 min. Upon completion the reaction is concentrated in vacuo to yield a residue. Et2O (15 mL) is added to the resultant solid/reside in order to (R)-1,1,1-trifluoropropan-2-d-2-amine HCl, which is filtered and washed with Et2O (2×5 mL) to yield (R)-1,1,1-trifluoropropan-2-d-2-amine HCl as a solid with an expected 92% ee.

### Preparation of (R)-1,1,1,1-trifluoropropan-3,3,3-d3-2-amine (Partially Deuterated)

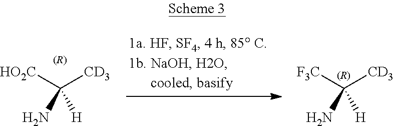

Step A: (R)-1,1,1-trifluoropropan-3,3,3-d3-2-amine. Commercially available D-alanine-3,3,3-d3 may be converted to (R)-1,1,1-trifluoropropan-3,3,3-d3-2-amine in accordance with methods known to those skilled in the art, for example as described in CN02875270 and DE3900300.

100 g (1.12 mol) of D-alanine-3,3,3-d3 are reacted with 280 g of sulfur tetrafluoride and 140 ml of HF in a stirred autoclave at 120° C. under internal pressure (about 21 bar) for 8 hours. After distilling off the volatile constituents, the residue is taken up in 500 ml of water, made alkaline with 45% strength sodium hydroxide solution, and the product is subsequently separated by steam distillation. The water vapor distillate is acidified with concentrated hydrochloric acid. After concentration and drying, (R)-1,1,1-trifluoropropan-3,3,3-d3-2-amine is isolated as the hydrochloride.

### Preparation of (R)-1,1,1-trifluoropropan-2,3,3,3-d4-2-amine (Fully Deuterated)

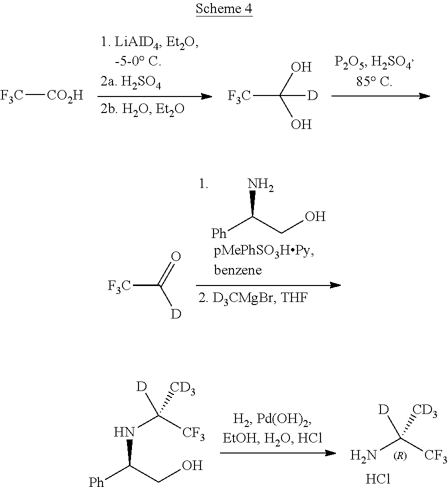

Step A: 2,2,2-trifluoroethane-1-d-1,1-diol. A solution of one mole of the perfluoroacetic acid in 1 liter of anhydrous ether is cooled to −5° C. (brine-bath) in a 3 liter flask fitted with addition funnel, stirrer and condenser. The system is flushed with nitrogen while cooling. A slurry of 21.5 g. of lithium aluminum deuteride in 750 ml. of anhydrous ether is added slowly with continuous stirring at −5° to 0° C. during 1.5 hours. Stirring is continued at −5° for one hour.

The reaction mixture is hydrolyzed with 40 ml. of water followed by 80 ml. of concentrated sulfuric acid in 200 ml. of water. The ether is decanted, and the solids remaining in the flask are dissolved in 300 ml. of water. The aqueous solution is extracted with ether, and the extracts are combined with the main ether portion and fractionally distilled to remove the solvent and alcohol leaving as a residue the crude aldehyde hydrate.

Step B: 2,2,2-trifluoroacetaldehyde-d. The crude aldehyde hydrate obtained in Step A is dropped slowly into a vigorously stirred mixture of phosphorus pentoxide and concentrated sulfuric acid heated to 85-90° C. The free aldehyde is collected in a suitably cooled receiver.

Step C: (R)-2-phenyl-2-(((R)-1,1,1-trifluoropropan-2-yl-2,3,3,3-d4)amino)ethan-1-ol. A mixture of 2,2,2-trifluoroacetaldehyde-d, (R)-2-amino-2-phenylethan-1-ol, and pyridinium p-toluenesulfonate (PPTS) is heated in benzene under reflux with azeotropic removal of water until no further water is removed to obtain (4R)-4-phenyl-2-(trifluoromethyl)oxazolidine. The chiral product is reacted with (methyl-d3)magnesium bromide to afford (R)-2-phenyl-2-(((R)-1,1,1-trifluoropropan-2-yl-2,3,3,3-d4)amino)ethan-1-ol. The chiral product may be further enriched for the desired stereoisomer by a person of ordinary skill via methods known in the art, such as high-performance liquid chromatography with a chiral chromatographic medium, for example.

Step D: (R)-1,1,1,-trifluoropropan-2,3,3,3-d4-2-amine. Hydrogenolysis of (R)-2-phenyl-2-(((R)-1,1,1-trifluoropropan-2-yl-2,3,3,3-d4)amino)ethan-1-ol (using H₂ and Pd(OH)₂ in an acidic ethanol/water solvent mixture) affords (R)-1,1,1-trifluoropropan-2,3,3,3-d4-2-amine.

### Preparation of Methyl 6-chloropicolinate-3,4,5-d3

Fully deuterated methyl 6-chloropicolinate may be prepared in accordance with WO2012168350 and WO2012049277. The procedures described therein would allow the skilled artisan to prepare compounds of formula (I) in which R₁, R₂, and R₃ are D, as shown below.

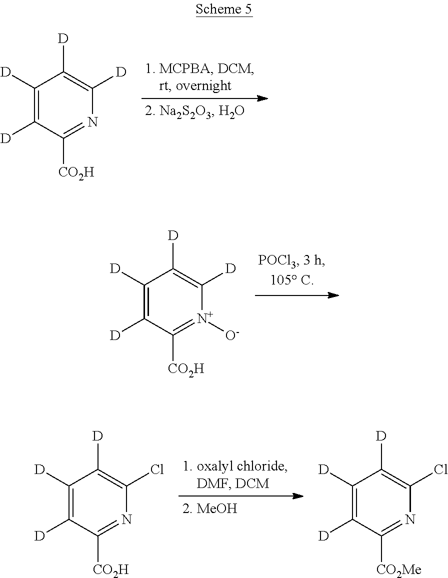 [SMILES: [2H]c1nc(C(=O)O)c([2H])c([2H])c1[2H] ; [2H]c1c([2H])c([2H])[n+]([O-])c(C(=O)O)c1[2H] ; [2H]c1c(Cl)nc(C(=O)O)c([2H])c1[2H] ; [2H]c1c(Cl)nc(C(=O)OC)c([2H])c1[2H]]

Step A: 2-Carboxypyridine 1-oxide-3,4,5,6-d4. Picolinic-3,4,5,6-d4 acid is commercially available. 5.0 g (29.2 mmol) of meta-chloroperoxybenzoic acid (m-CPBA) is added to a solution of picolinic-3,4,5,6-d4 acid (14.6 mmol) in methylene chloride (50 mL) and the mixture is stirred overnight at room temperature. The solid is filtered off, quenched with a saturated solution of sodium thio sulfate (50 mL), and the mixture is extracted with methylene chloride (3×60 mL). The organic layers are combined, dried over anhydrous sodium sulfate and concentrated in vacuo to give a solid which is washed with ether (5×20 mL) to afford 2-Carboxypyridine 1-oxide-3,4,5,6-d4.

Step B: 6-chloropicolinic-3,4,5-d3 acid. 2-Carboxypyridine 1-oxide-3,4,5,6-d4 (62 mmol) is added into POCl₃ (10 g) at 0° C. and the mixture is heated to at 95-105° C. for 1-3 hrs. The reaction mixture is evaporated to dryness. The residue is dissolved in 15 mL water and extracted with ethyl acetate (2×15 mL), the combined organic layer is washed with water (2×30 mL) and brine (30 mL), then evaporated to dryness to obtain the title compound.

Step C. methyl 6-chloropicolinate-3,4,5-d3. 6-Chloropicolinic-3,4,5-d3 acid (31.7 mmol) is dissolved in DCM (150 mL) and oxalyl chloride (5.45 mL, 63.5 mmol) and DMF (1 mL) are added. The reaction mixture is stirred for 3 h, concentrated in vacuo and azeotroped with DCM. The residue is dissolved in THF (150 mL) and potassium tert-butoxide (3.39 mg, 47.6 mmol) is added. The reaction mixture is stirred for 18 h, quenched with water (250 mL) and extracted with DCM (3×150 mL). The combined organic fractions are washed with sat aq NaHCO₃(150 mL), dried (MgSO₄) and concentrated in vacuo. The residue is purified by column chromatography to give the title compound.

Similar methods can be used to prepare partially deuterated methyl 6-chloropicolinates by using partially deuterated picolinic acid starting materials (e.g., Compounds A and B), as illustrated below.

### Availability and Preparation of Undeuterated and Partially Methyl 6-Chloropicolinates

Methyl 6-chloropicolinate is commercially available and may be used to introduce an undeuterated ring to compounds of formula (I) such that compounds in which R₁, R₂, and R₃ are H are obtained.

Partially deuterated methyl 6-chloropicolinate, i.e. containing one to three deuterium atoms on the pyridine ring, can be prepared according to other methods and procedures found in the scientific literature and known to one of skill in the art. For example, compound A is commercially available and may be used to produce compounds of formula (I) that are doubly deuterated at the R₁ and R₂ positions on the pyridinyl ring system.

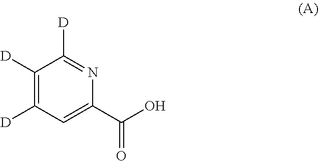 [SMILES: [2H]c1cc(C(=O)O)nc([2H])c1[2H]]

Additionally, compound B may be prepared in accordance with Spitzner D, Science of Synthesis, Vol. 15, pp. 11-284, (2005). Compound B may be used to produce compounds of formula (I) that are singly deuterated at the R₃ position on the pyridinyl ring system.

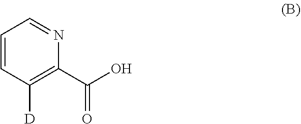

### Preparation of Partially or Fully Deuterated Compound of Formula (I)

A partially or fully deuterated compound of formula (I) may be prepared in accordance with the following scheme. Any combination of undeuterated, partially deuterated, or fully deuterated methyl 6-chloropicolinate and any undeuterated or partially or fully deuterated (R)-1,1,1-trifluoropropan-2-amine hydrochloride may be used to prepare compounds of formula (I) with the desired deuteration level. The starting materials employed in the reactions shown in the scheme (i.e., (R)-1,1,1-Trifluoropropan-2-d-2-amines and methyl 6-chloropicolinates) determine the location of the deuterium atoms in the resulting compound of formula (I).

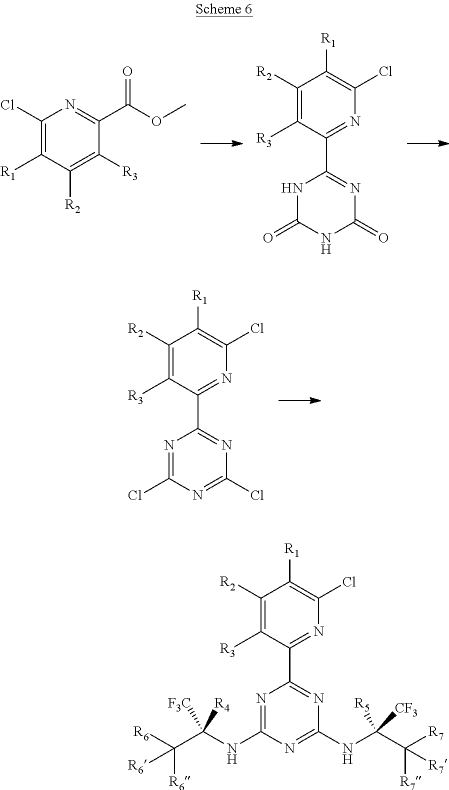

wherein R₁, R₂, R₃, R₄, R₅, R₆, R_{6′}, R_{6″}, R₇, R_{7′} and R_{7″} are as defined above for various embodiments.

Step 1: Preparation of Undeuterated or Partially or Fully Deuterated 6-(6-chloropyridin-2-yl)-1,3,5-triazine-2,4(1H,3H)-dione. To a dried three-necked round bottom flask are added biuret (14.8 g, 0.14 mol) and undeuterated or partially or fully deuterated methyl 6-chloropicolinate (0.12 mol) and EtOH (250 mL). The mixture is degassed with N₂ three times and then stirred at 25° C. for 20 min. Then the temperature is allowed to rise to 50° C., followed by addition of HC(OMe)₃ (17 mL, 0.14 mol) and TFA (1.37 g, 0.01 mol). The reaction mixture is stirred at this temperature for 30 min, followed by dropwise addition of a solution of NaOEt in EtOH (20% wt, 163 g, 0.48 mol). The resulting mixture/slurry is heated to reflux for 2 hr until the reaction is complete. The mixture is cooled to r.t. and concentrated under reduced pressure. The residue is treated with water (200 mL) and concentrated under reduced pressure to remove the remaining ethanol. Then water (300 mL) is added to the residue (while stirring) to form a clear solution. The solution is cooled to 10° C. and slowly adjusted to pH 1 by 6N HCl. The resulting mixture is stirred for another 2 hr and filtered. The filter cake is washed with aq. HCl (pH=1), collected and suspended in DCM (300 mL). The suspension is stirred at r.t. for 2 hr, filtered and dried to afford the desired product.

Step 2: Preparation of Undeuterated or Partially or Fully Deuterated 2,4-dichloro-6-(6-chloropyridin-2-yl)-1,3,5-triazine. To a solution of an undeuterated or partially or fully deuterated 6-(6-chloropyridin-2-yl)-1,3,5-triazine-2,4(1H,3H)-dione (0.013 mol) in POCl₃ (48 mL) is added PCl₅ (23 g, 0.1 mol). The mixture is stirred at 100° C. for 2 hr and then concentrated. The residue is dissolved in EtOAc and then washed with Sat. aq. NaHCO₃. The organic layer is dried over anhydrous Na₂SO₄ and then concentrated to give the desired product.

Step 3: Preparation of Undeuterated or Partially or Fully Deuterated 6-(6-chloropyridin-2-yl)-N²,N⁴-bis((R)-1,1,1-trifluoro propan-2-yl)-1,3,5-triazine-2,4-diamine. A mixture of undeuterated or partially or fully deuterated 2,4-dichloro-6-(6-chloro-pyridin-2-yl)-1,3,5-triazine (1.04 mol), undeuterated or partially or fully deuterated (R)-1,1-trifluoropropan-2-amine hydrochloride (0.39 g, 2.6 mol), and potassium carbonate (0.43 g, 3.1 mol) in dry 1,4-dioxane (2.5 mL) is stirred under the atmosphere of N₂ at 50° C. for 36 hr then at 100° C. for another 36 hr until the reaction is complete. The resulting mixture is filtered through Celite and the cake is washed with EtOAc. The filtrate is concentrated and the residue is purified by standard methods to give the desired product.

A person of ordinary skill in the art would be able to prepare partially deuterated compounds of formula (I) via the teachings disclosed herein together with methods known in the art.

Using the procedures described above alone or in combination with other procedures adapted from methods known in the art, the following compounds listed in Table 1, or pharmaceutically acceptable salts or co-crystalline materials thereof, may be prepared:

TABLE 1

| Compound | | |
|---|---|---|
| Number | Structure | Compound Name |
| 1 | 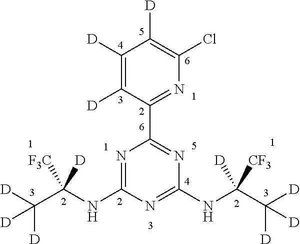 [SMILES: [2H]c1c(Cl)nc(-c2nc(N[C@]([2H])(C([2H])([2H])[2H])C(F)(F)F)nc(N[C@]([2H])(C([2H])([2H])[2H])C(F)(F)F)n2)c([2H])c1[2H]] | 6-(6-chloropyridin-2-yl-3,4,5-d3)- N2,N4-bis((R)-1,1,1- trifluoropropan-2-yl-2,3,3,3-d4)- 1,3,5-triazine-2,4-diamine |
| 2 | 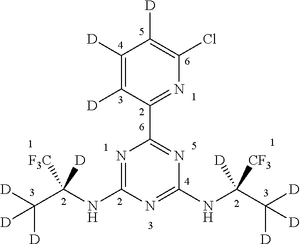 [SMILES: [2H]c1c(Cl)nc(-c2nc(N[C@]([2H])(C([2H])([2H])[2H])C(F)(F)F)nc(N[C@]([2H])(C([2H])([2H])[2H])C(F)(F)F)n2)c([2H])c1[2H]] | 6-(6-chloropyridin-2-yl-3,4,5-d3)- N2-((R)-1,1,1-trifluoropropan-2-yl- 2,3,3,3-d4)-N4-((R)-1,1,1- trifluoropropan-2-yl-3,3,3-d3)- 1,3,5-triazine-2,4-diamine |
| 3 | 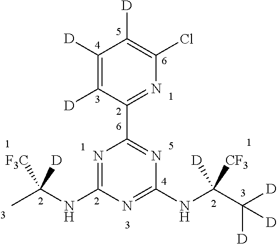 [SMILES: [2H]c1c(Cl)nc(-c2nc(N[C@]([2H])(C([2H])([2H])[2H])C(F)(F)F)nc(N[C@]([2H])(C)C(F)(F)F)n2)c([2H])c1[2H]] | 6-(6-chloropyridin-2-yl-3,4,5-d3)- N2-((R)-1,1,1-trifluoropropan-2-yl- 2-d)-N4-((R)-1,1,1- trifluoropropan-2-yl-3,3,3-d3)- 1,3,5-triazine-2,4-diamine |
| 4 | 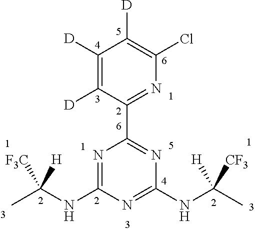 [SMILES: [2H]c1c(Cl)nc(-c2nc(N[C@H](C)C(F)(F)F)nc(N[C@H](C)C(F)(F)F)n2)c([2H])c1[2H]] | 6-(6-chloropyridin-2-yl-3,4,5-d3)- N2,N4-bis((R)-1,1,1- trifluoropropan-2-yl)-1,3,5- triazine-2,4-diamine |
| 5 | 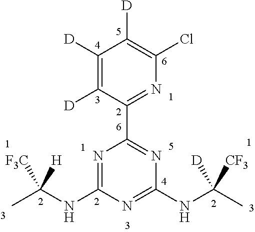 [SMILES: [2H]c1c(Cl)nc(-c2nc(N[C@H](C)C(F)(F)F)nc(N[C@]([2H])(C)C(F)(F)F)n2)c([2H])c1[2H]] | 6-(6-chloropyridin-2-yl-3,4,5-d3)- N2-((R)-1,1,1-trifluoropropan-2- yl)-N4-((R)-1,1,1-trifluoropropan- 2-yl-2-d)-1,3,5-triazine-2,4- diamine |
| 6 | 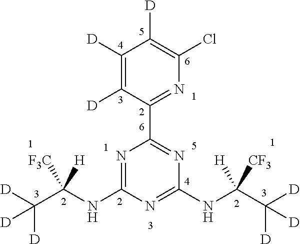 [SMILES: [2H]c1c(Cl)nc(-c2nc(N[C@H](C([2H])([2H])[2H])C(F)(F)F)nc(N[C@H](C([2H])([2H])[2H])C(F)(F)F)n2)c([2H])c1[2H]] | 6-(6-chloropyridin-2-yl-3,4,5-d3)- N2,N4-bis((R)-1,1,1- trifluoropropan-2-yl-3,3,3-d3)- 1,3,5-triazine-2,4-diamine |
| 7 | 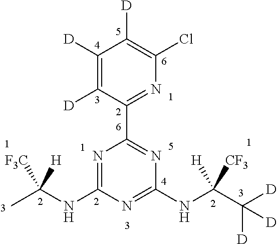 [SMILES: [2H]c1c(Cl)nc(-c2nc(N[C@H](C([2H])([2H])[2H])C(F)(F)F)nc(N[C@H](C)C(F)(F)F)n2)c([2H])c1[2H]] | 6-(6-chloropyridin-2-yl-3,4,5-d3)- N2-((R)-1,1,1-trifluoropropan-2- yl)-N4-((R)-1,1,1-trifluoropropan- 2-yl-3,3,3-d3)-1,3,5-triazine-2,4- diamine |
| 8 | 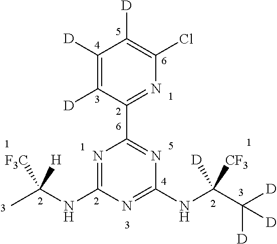 [SMILES: [2H]c1c(Cl)nc(-c2nc(N[C@H](C)C(F)(F)F)nc(N[C@]([2H])(C([2H])([2H])[2H])C(F)(F)F)n2)c([2H])c1[2H]] | 6-(6-chloropyridin-2-yl-3,4,5-d3)- N2-((R)-1,1,1-trifluoropropan-2- yl)-N4-((R)-1,1,1-trifluoropropan- 2-yl-2,3,3,3-d4)-1,3,5-triazine-2,4- diamine |
| 9 | 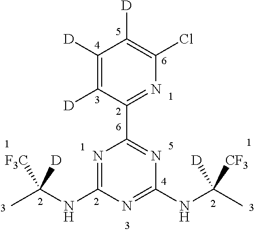 [SMILES: [2H]c1c(Cl)nc(-c2nc(N[C@]([2H])(C)C(F)(F)F)nc(N[C@]([2H])(C)C(F)(F)F)n2)c([2H])c1[2H]] | 6-(6-chloropyridin-2-yl-3,4,5-d3)- N2,N4-bis((R)-1,1,1- trifluoropropan-2-yl-2-d)-1,3,5- triazine-2,4-diamine |
| 10 | 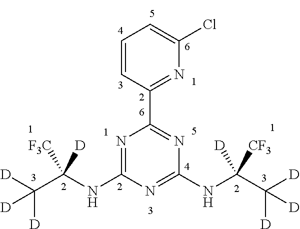 [SMILES: [2H]C([2H])([2H])[C@@]([2H])(Nc1nc(N[C@]([2H])(C([2H])([2H])[2H])C(F)(F)F)nc(-c2cccc(Cl)n2)n1)C(F)(F)F] | 6-(6-chloropyridin-2-yl)-N2,N4- bis((R)-1,1,1-trifluoropropan-2-yl- 2,3,3,3-d4)-1,3,5-triazine-2,4- diamine |
| 11 | 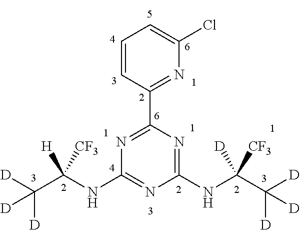 [SMILES: [2H]C([2H])([2H])[C@@H](Nc1nc(N[C@]([2H])(C([2H])([2H])[2H])C(F)(F)F)nc(-c2cccc(Cl)n2)n1)C(F)(F)F] | 6-(6-chloropyridin-2-yl)-N2-((R)- 1,1,1-trifluoropropan-2-yl-2,3,3,3- d4)-N4-((R)-1,1,1-trifluoropropan- 2-yl-3,3,3-d3)-1,3,5-triazine-2,4- diamine |
| 12 | 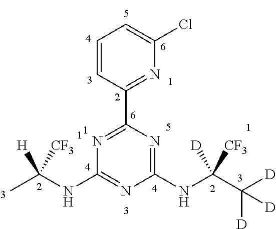 [SMILES: [2H]C([2H])([2H])[C@@]([2H])(Nc1nc(N[C@H](C)C(F)(F)F)nc(-c2cccc(Cl)n2)n1)C(F)(F)F] | 6-(6-chloropyridin-2-yl)-N2-((R)- 1,1,1-trifluoropropan-2-yl)-N4- ((R)-1,1,1-trifluoropropan-2-yl- 2,3,3,3-d4)-1,3,5-triazine-2,4- diamine |
| 13 | 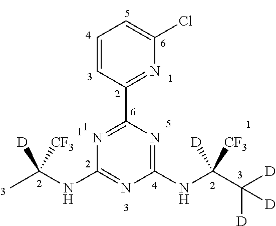 [SMILES: [2H]C([2H])([2H])[C@@]([2H])(Nc1nc(N[C@]([2H])(C)C(F)(F)F)nc(-c2cccc(Cl)n2)n1)C(F)(F)F] | 6-(6-chloropyridin-2-yl)-N2-((R)- 1,1,1-trifluoropropan-2-yl-2-d)-N4- ((R)-1,1,1-trifluoropropan-2-yl- 3,3,3-d3)-1,3,5-triazine-2,4- diamine |
| 14 | 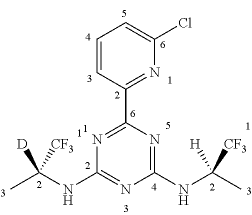 [SMILES: [2H][C@](C)(Nc1nc(N[C@H](C)C(F)(F)F)nc(-c2cccc(Cl)n2)n1)C(F)(F)F] | 6-(6-chloropyridin-2-yl)-N2-((R)- 1,1,1-trifluoropropan-2-yl)-N4- ((R)-1,1,1-trifluoropropan-2-yl-2- d)-1,3,5-triazine-2,4-diamine |
| 15 | 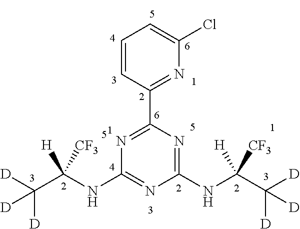 [SMILES: [2H]C([2H])([2H])[C@@H](Nc1nc(N[C@H](C([2H])([2H])[2H])C(F)(F)F)nc(-c2cccc(Cl)n2)n1)C(F)(F)F] | 6-(6-chloropyridin-2-yl)-N2,N4- bis((R)-1,1,1-trifluoropropan-2-yl- 3,3,3-d3)-1,3,5-triazine-2,4- diamine |
| 16 | 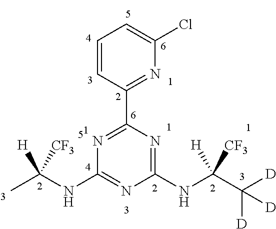 [SMILES: [2H]C([2H])([2H])[C@@H](Nc1nc(N[C@H](C)C(F)(F)F)nc(-c2cccc(Cl)n2)n1)C(F)(F)F] | 6-(6-chloropyridin-2-yl)-N2-((R)- 1,1,1-trifluoropropan-2-yl)-N4- ((R)-1,1,1-trifluoropropan-2-yl- 3,3,3-d3)-1,3,5-triazine-2,4- diamine |
| 17 | 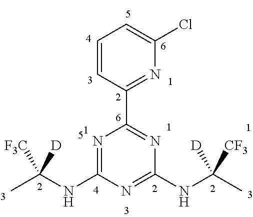 [SMILES: [2H][C@](C)(Nc1nc(N[C@]([2H])(C)C(F)(F)F)nc(-c2cccc(Cl)n2)n1)C(F)(F)F] | 6-(6-chloropyridin-2-yl)-N2,N4- bis((R)-1,1,1-trifluoropropan-2-yl- 2-d)-1,3,5-triazine-2,4-diamine |
| 18 | 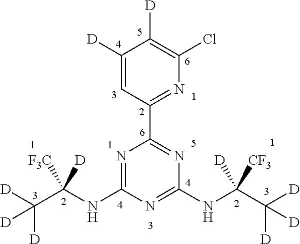 [SMILES: [2H]c1cc(-c2nc(N[C@]([2H])(C([2H])([2H])[2H])C(F)(F)F)nc(N[C@]([2H])(C([2H])([2H])[2H])C(F)(F)F)n2)nc(Cl)c1[2H]] | 6-(6-chloropyridin-2-yl-4,5-d2)- N2,N4-bis((R)-1,1,1- trifluoropropan-2-yl-2,3,3,3-d4)- 1,3,5-triazine-2,4-diamine |
| 19 | 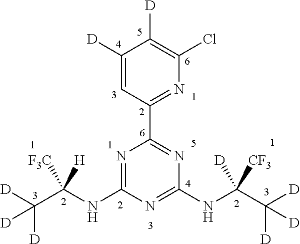 [SMILES: [2H]c1cc(-c2nc(N[C@H](C([2H])([2H])[2H])C(F)(F)F)nc(N[C@]([2H])(C([2H])([2H])[2H])C(F)(F)F)n2)nc(Cl)c1[2H]] | 6-(6-chloropyridin-2-yl-4,5-d2)- N2-((R)-1,1,1-trifluoropropan-2-yl- 2,3,3,3-d4)-N4-((R)-1,1,1- trifluoropropan-2-yl-3,3,3-d3)- 1,3,5-triazine-2,4-diamine |
| 20 | 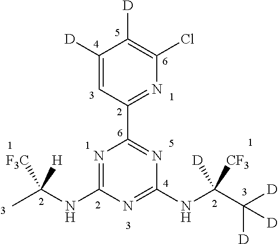 [SMILES: [2H]c1cc(-c2nc(N[C@H](C)C(F)(F)F)nc(N[C@]([2H])(C([2H])([2H])[2H])C(F)(F)F)n2)nc(Cl)c1[2H]] | 6-(6-chloropyridin-2-yl-4,5-d2)- N2-((R)-1,1,1-trifluoropropan-2- yl)-N4-((R)-1,1,1-trifluoropropan- 2-yl-3,3,3-d3)-1,3,5-triazine-2,4- diamine |
| 21 | 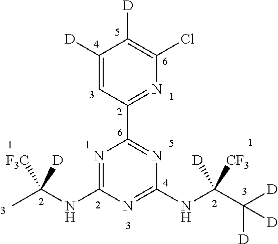 [SMILES: [2H]c1cc(-c2nc(N[C@]([2H])(C([2H])([2H])[2H])C(F)(F)F)nc(N[C@]([2H])(C)C(F)(F)F)n2)nc(Cl)c1[2H]] | 6-(6-chloropyridin-2-yl-4,5-d2)- N2-((R)-1,1,1-trifluoropropan-2-yl- 2,3,3,3-d4)-N4-((R)-1,1,1- trifluoropropan-2-yl-2-d)-1,3,5- triazine-2,4-diamine |
| 22 | 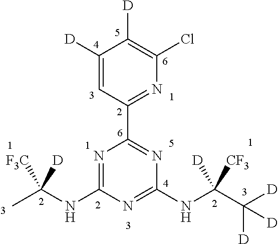 [SMILES: [2H]c1cc(-c2nc(N[C@]([2H])(C([2H])([2H])[2H])C(F)(F)F)nc(N[C@]([2H])(C)C(F)(F)F)n2)nc(Cl)c1[2H]] | 6-(6-chloropyridin-2-yl-4,5-d2)- N2,N4-bis((R)-1,1,1- trifluoropropan-2-yl-2-d)-1,3,5- triazine-2,4-diamine |
| 23 | 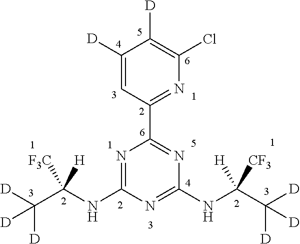 [SMILES: [2H]c1cc(-c2nc(N[C@H](C([2H])([2H])[2H])C(F)(F)F)nc(N[C@H](C([2H])([2H])[2H])C(F)(F)F)n2)nc(Cl)c1[2H]] | 6-(6-chloropyridin-2-yl-4,5-d2)- N2,N4-bis((R)-1,1,1- trifluoropropan-2-yl-3,3,3-d3)- 1,3,5-triazine-2,4-diamine |
| 24 | 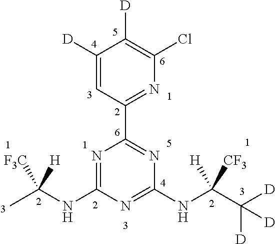 [SMILES: [2H]c1cc(-c2nc(N[C@H](C([2H])([2H])[2H])C(F)(F)F)nc(N[C@H](C)C(F)(F)F)n2)nc(Cl)c1[2H]] | 6-(6-chloropyridin-2-yl-4,5-d2)- N2-((R)-1,1,1-trifluoropropan-2- yl)-N4-((R)-1,1,1-trifluoropropan- 2-yl-2,3,3,3-d4)-1,3,5-triazine-2,4- diamine |
| 25 | 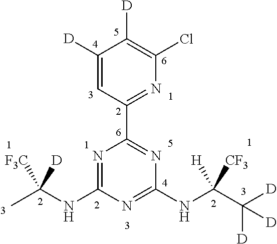 [SMILES: [2H]c1cc(-c2nc(N[C@H](C([2H])([2H])[2H])C(F)(F)F)nc(N[C@]([2H])(C)C(F)(F)F)n2)nc(Cl)c1[2H]] | 6-(6-chloropyridin-2-yl-4,5-d2)- N2-((R)-1,1,1-trifluoropropan-2-yl- 2-d)-N4-((R)-1,1,1- trifluoropropan-2-yl-3,3,3-d3)- 1,3,5-triazine-2,4-diamine |
| 26 | 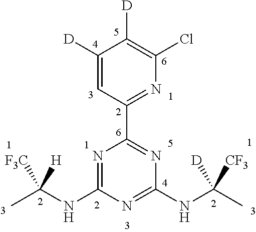 [SMILES: [2H]c1cc(-c2nc(N[C@H](C)C(F)(F)F)nc(N[C@]([2H])(C)C(F)(F)F)n2)nc(Cl)c1[2H]] | 6-(6-chloropyridin-2-yl-4,5-d2)- N2-((R)-1,1,1-trifluoropropan-2- yl)-N4-((R)-1,1,1-trifluoropropan- 2-yl-2-d)-1,3,5-triazine-2,4- diamine |
| 27 | 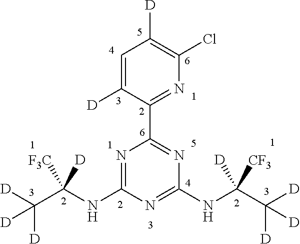 [SMILES: [2H]c1cc([2H])c(-c2nc(N[C@]([2H])(C([2H])([2H])[2H])C(F)(F)F)nc(N[C@]([2H])(C([2H])([2H])[2H])C(F)(F)F)n2)nc1Cl] | 6-(6-chloropyridin-2-yl-3,5-d2)- N2,N4-bis((R)-1,1,1- trifluoropropan-2-yl-2,3,3,3-d4)- 1,3,5-triazine-2,4-diamine |
| 28 | 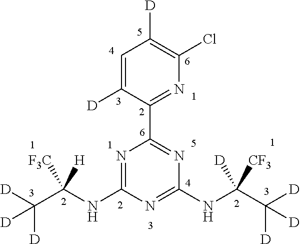 [SMILES: [2H]c1cc([2H])c(-c2nc(N[C@H](C([2H])([2H])[2H])C(F)(F)F)nc(N[C@]([2H])(C([2H])([2H])[2H])C(F)(F)F)n2)nc1Cl] | 6-(6-chloropyridin-2-yl-3,5-d2)- N2-((R)-1,1,1-trifluoropropan-2-yl- 2,3,3,3-d4)-N4-((R)-1,1,1- trifluoropropan-2-yl-3,3,3-d3)- 1,3,5-triazine-2,4-diamine |
| 29 | 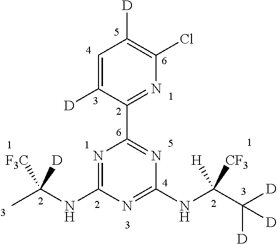 [SMILES: [2H]c1cc([2H])c(-c2nc(N[C@H](C([2H])([2H])[2H])C(F)(F)F)nc(N[C@]([2H])(C)C(F)(F)F)n2)nc1Cl] | 6-(6-chloropyridin-2-yl-3,5-d2)- N2-((R)-1,1,1-trifluoropropan-2-yl- 2-d)-N4-((R)-1,1,1- trifluoropropan-2-yl-3,3,3-d3)- 1,3,5-triazine-2,4-diamine |
| 30 | 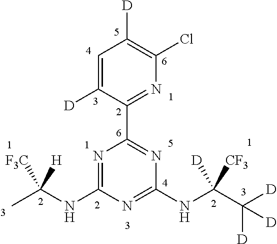 [SMILES: [2H]c1cc([2H])c(-c2nc(N[C@H](C)C(F)(F)F)nc(N[C@]([2H])(C([2H])([2H])[2H])C(F)(F)F)n2)nc1Cl] | 6-(6-chloropyridin-2-yl-3,5-d2)- N2-((R)-1,1,1-trifluoropropan-2- yl)-N4-((R)-1,1,1-trifluoropropan- 2-yl-2,3,3,3-d4)-1,3,5-triazine-2,4- diamine |
| 31 | 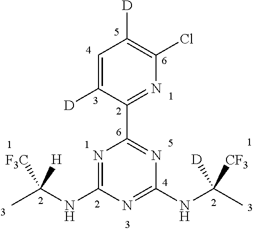 [SMILES: [2H]c1cc([2H])c(-c2nc(N[C@H](C)C(F)(F)F)nc(N[C@]([2H])(C)C(F)(F)F)n2)nc1Cl] | 6-(6-chloropyridin-2-yl-3,5-d2)- N2-((R)-1,1,1-trifluoropropan-2- yl)-N4-((R)-1,1,1-trifluoropropan- 2-yl-2-d)-1,3,5-triazine-2,4- diamine |
| 32 | 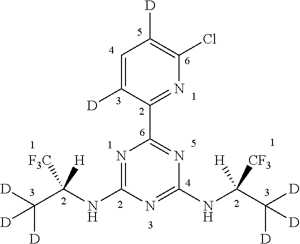 [SMILES: [2H]c1cc([2H])c(-c2nc(N[C@H](C([2H])([2H])[2H])C(F)(F)F)nc(N[C@H](C([2H])([2H])[2H])C(F)(F)F)n2)nc1Cl] | 6-(6-chloropyridin-2-yl-3,5-d2)- N2,N4-bis((R)-1,1,1- trifluoropropan-2-yl-3,3,3-d3)- 1,3,5-triazine-2,4-diamine |
| 33 | 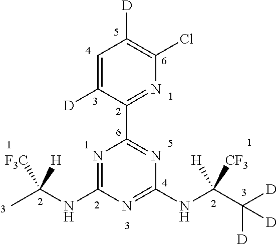 [SMILES: [2H]c1cc([2H])c(-c2nc(N[C@H](C([2H])([2H])[2H])C(F)(F)F)nc(N[C@H](C)C(F)(F)F)n2)nc1Cl] | 6-(6-chloropyridin-2-yl-3,5-d2)- N2-((R)-1,1,1-trifluoropropan-2- yl)-N4-((R)-1,1,1-trifluoropropan- 2-yl-3,3,3-d3)-1,3,5-triazine-2,4- diamine |
| 34 | 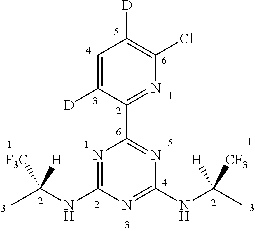 [SMILES: [2H]c1cc([2H])c(-c2nc(N[C@H](C)C(F)(F)F)nc(N[C@H](C)C(F)(F)F)n2)nc1Cl] | 6-(6-chloropyridin-2-yl-3,5-d2)- N2,N4-bis((R)-1,1,1- trifluoropropan-2-yl)-1,3,5- triazine-2,4-diamine |
| 35 | 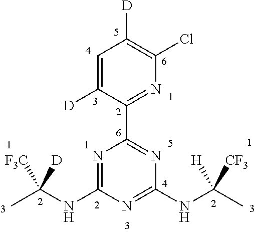 [SMILES: [2H]c1cc([2H])c(-c2nc(N[C@H](C)C(F)(F)F)nc(N[C@]([2H])(C)C(F)(F)F)n2)nc1Cl] | 6-(6-chloropyridin-2-yl-3,5-d2)- N2,N4-bis((R)-1,1,1- trifluoropropan-2-yl-2-d)-1,3,5- triazine-2,4-diamine |
| 36 | 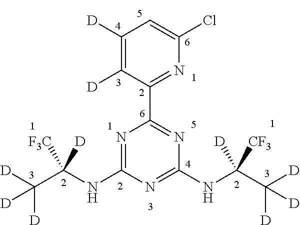 [SMILES: [2H]c1cc(Cl)nc(-c2nc(N[C@]([2H])(C([2H])([2H])[2H])C(F)(F)F)nc(N[C@]([2H])(C([2H])([2H])[2H])C(F)(F)F)n2)c1[2H]] | 6-(6-chloropyridin-2-yl-3,4-d2)- N2,N4-bis((R)-1,1,1- trifluoropropan-2-yl-2,3,3,3-d4)- 1,3,5-triazine-2,4-diamine |
| 37 | 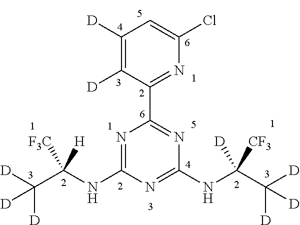 [SMILES: [2H]c1cc(Cl)nc(-c2nc(N[C@H](C([2H])([2H])[2H])C(F)(F)F)nc(N[C@]([2H])(C([2H])([2H])[2H])C(F)(F)F)n2)c1[2H]] | 6-(6-chloropyridin-2-yl-3,4-d2)- N2-((R)-1,1,1-trifluoropropan-2-yl- 2,3,3,3-d4)-N4-((R)-1,1,1- trifluoropropan-2-yl-3,3,3-d3)- 1,3,5-triazine-2,4-diamine |
| 38 | 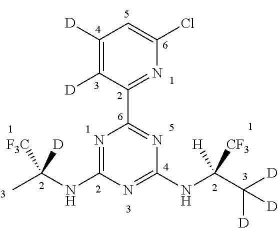 [SMILES: [2H]c1cc(Cl)nc(-c2nc(N[C@H](C([2H])([2H])[2H])C(F)(F)F)nc(N[C@]([2H])(C)C(F)(F)F)n2)c1[2H]] | 6-(6-chloropyridin-2-yl-3,4-d2)- N2-((R)-1,1,1-trifluoropropan-2-yl- 2-d)-N4-((R)-1,1,1- trifluoropropan-2-yl-3,3,3-d3)- 1,3,5-triazine-2,4-diamine |
| 39 | 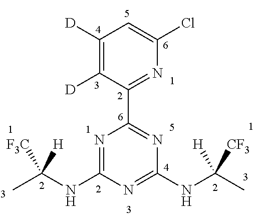 [SMILES: [2H]c1cc(Cl)nc(-c2nc(N[C@H](C)C(F)(F)F)nc(N[C@H](C)C(F)(F)F)n2)c1[2H]] | 6-(6-chloropyridin-2-yl-3,4-d2)- N2,N4-bis((R)-1,1,1- trifluoropropan-2-yl)-1,3,5- triazine-2,4-diamine |
| 40 | 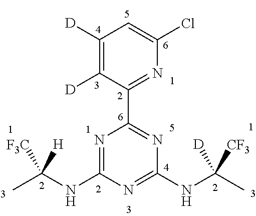 [SMILES: [2H]c1cc(Cl)nc(-c2nc(N[C@H](C)C(F)(F)F)nc(N[C@]([2H])(C)C(F)(F)F)n2)c1[2H]] | 6-(6-chloropyridin-2-yl-3,4-d2)- N2-((R)-1,1,1-trifluoropropan-2- yl)-N4-((R)-1,1,1-trifluoropropan- 2-yl-2-d)-1,3,5-triazine-2,4- diamine |
| 41 | 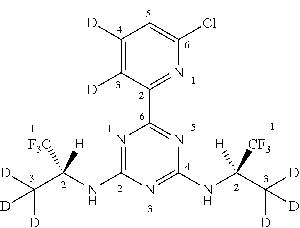 [SMILES: [2H]c1cc(Cl)nc(-c2nc(N[C@H](C([2H])([2H])[2H])C(F)(F)F)nc(N[C@H](C([2H])([2H])[2H])C(F)(F)F)n2)c1[2H]] | 6-(6-chloropyridin-2-yl-3,4-d2)- N2,N4-bis((R)-1,1,1- trifluoropropan-2-yl-3,3,3-d3)- 1,3,5-triazine-2,4-diamine |
| 42 | 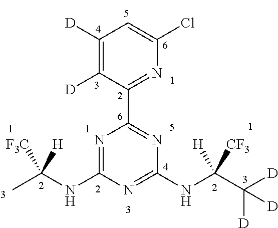 [SMILES: [2H]c1cc(Cl)nc(-c2nc(N[C@H](C([2H])([2H])[2H])C(F)(F)F)nc(N[C@H](C)C(F)(F)F)n2)c1[2H]] | 6-(6-chloropyridin-2-yl-3,4-d2)- N2-((R)-1,1,1-trifluoropropan-2- yl)-N4-((R)-1,1,1-trifluoropropan- 2-yl-3,3,3-d3)-1,3,5-triazine-2,4- diamine |
| 43 | 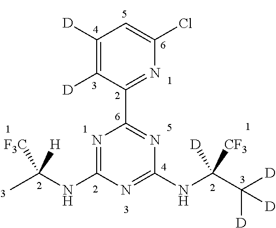 [SMILES: [2H]c1cc(Cl)nc(-c2nc(N[C@H](C)C(F)(F)F)nc(N[C@]([2H])(C([2H])([2H])[2H])C(F)(F)F)n2)c1[2H]] | 6-(6-chloropyridin-2-yl-3,4-d2)- N2-((R)-1,1,1-trifluoropropan-2- yl)-N4-((R)-1,1,1-trifluoropropan- 2-yl-2,3,3,3-d4)-1,3,5-triazine-2,4- diamine |
| 44 | 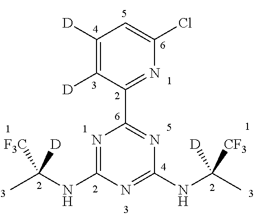 [SMILES: [2H]c1cc(Cl)nc(-c2nc(N[C@]([2H])(C)C(F)(F)F)nc(N[C@]([2H])(C)C(F)(F)F)n2)c1[2H]] | 6-(6-chloropyridin-2-yl-3,4-d2)- N2,N4-bis((R)-1,1,1- trifluoropropan-2-yl-2-d)-1,3,5- triazine-2,4-diamine |
| 45 | 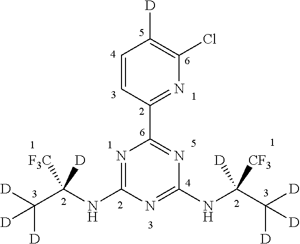 [SMILES: [2H]c1ccc(-c2nc(N[C@]([2H])(C([2H])([2H])[2H])C(F)(F)F)nc(N[C@]([2H])(C([2H])([2H])[2H])C(F)(F)F)n2)nc1Cl] | 6-(6-chloropyridin-2-yl-5-d)- N2,N4-bis((R)-1,1,1- trifluoropropan-2-yl-2,3,3,3-d4)- 1,3,5-triazine-2,4-diamine |
| 46 | 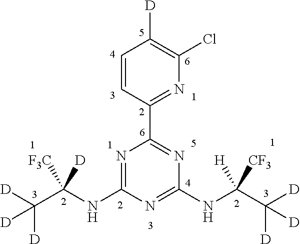 [SMILES: [2H]c1ccc(-c2nc(N[C@H](C([2H])([2H])[2H])C(F)(F)F)nc(N[C@]([2H])(C([2H])([2H])[2H])C(F)(F)F)n2)nc1Cl] | 6-(6-chloropyridin-2-yl-5-d)-N2- ((R)-1,1,1-trifluoropropan-2-yl- 2,3,3,3-d4)-N4-((R)-1,1,1- trifluoropropan-2-yl-3,3,3-d3)- 1,3,5-triazine-2,4-diamine |
| 47 | 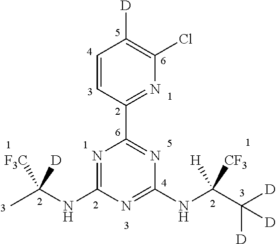 [SMILES: [2H]c1ccc(-c2nc(N[C@H](C([2H])([2H])[2H])C(F)(F)F)nc(N[C@]([2H])(C)C(F)(F)F)n2)nc1Cl] | 6-(6-chloropyridin-2-yl-5-d)-N2- ((R)-1,1,1-trifluoropropan-2-yl-2- d)-N4-((R)-1,1,1-trifluoropropan- 2-yl-3,3,3-d3)-1,3,5-triazine-2,4- diamine |
| 48 | 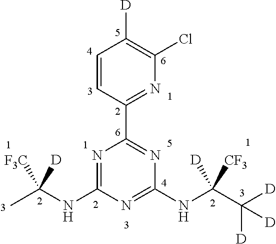 [SMILES: [2H]c1ccc(-c2nc(N[C@]([2H])(C([2H])([2H])[2H])C(F)(F)F)nc(N[C@]([2H])(C)C(F)(F)F)n2)nc1Cl] | 6-(6-chloropyridin-2-yl-5-d)-N2- ((R)-1,1,1-trifluoropropan-2-yl- 2,3,3,3-d4)-N4-((R)-1,1,1- trifluoropropan-2-yl-2-d)-1,3,5- triazine-2,4-diamine |
| 49 | 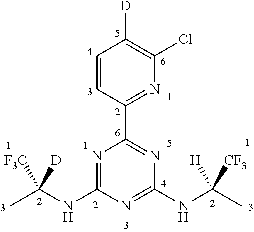 [SMILES: [2H]c1ccc(-c2nc(N[C@H](C)C(F)(F)F)nc(N[C@]([2H])(C)C(F)(F)F)n2)nc1Cl] | 6-(6-chloropyridin-2-yl-5-d)-N2- ((R)-1,1,1-trifluoropropan-2-yl)- N4-((R)-1,1,1-trifluoropropan-2-yl- 2-d)-1,3,5-triazine-2,4-diamine |
| 50 | 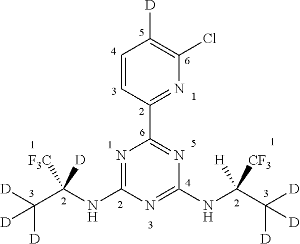 [SMILES: [2H]c1ccc(-c2nc(N[C@H](C([2H])([2H])[2H])C(F)(F)F)nc(N[C@]([2H])(C([2H])([2H])[2H])C(F)(F)F)n2)nc1Cl] | 6-(6-chloropyridin-2-yl-5-d)- N2,N4-bis((R)-1,1,1- trifluoropropan-2-yl-3,3,3-d3)- 1,3,5-triazine-2,4-diamine |
| 51 | 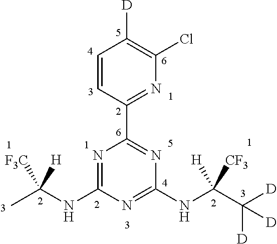 [SMILES: [2H]c1ccc(-c2nc(N[C@H](C([2H])([2H])[2H])C(F)(F)F)nc(N[C@H](C)C(F)(F)F)n2)nc1Cl] | 6-(6-chloropyridin-2-yl-5-d)-N2- ((R)-1,1,1-trifluoropropan-2-yl)- N4-((R)-1,1,1-trifluoropropan-2-yl- 3,3,3-d3)-1,3,5-triazine-2,4- diamine |
| 52 | 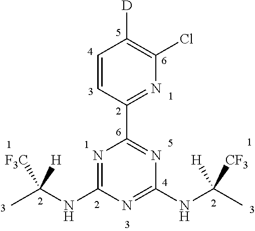 [SMILES: [2H]c1ccc(-c2nc(N[C@H](C)C(F)(F)F)nc(N[C@H](C)C(F)(F)F)n2)nc1Cl] | 6-(6-chloropyridin-2-yl-5-d)- N2,N4-bis((R)-1,1,1- trifluoropropan-2-yl)-1,3,5- triazine-2,4-diamine |
| 53 | 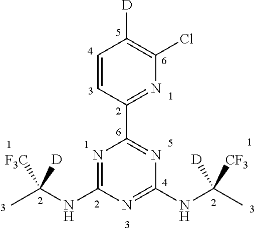 [SMILES: [2H]c1ccc(-c2nc(N[C@]([2H])(C)C(F)(F)F)nc(N[C@]([2H])(C)C(F)(F)F)n2)nc1Cl] | 6-(6-chloropyridin-2-yl-5-d)- N2,N4-bis((R)-1,1,1- trifluoropropan-2-yl-2-d)-1,3,5- triazine-2,4-diamine |
| 54 | 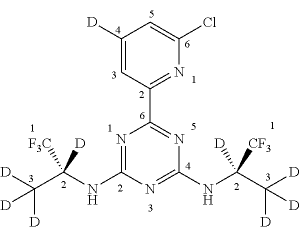 [SMILES: [2H]c1cc(Cl)nc(-c2nc(N[C@]([2H])(C([2H])([2H])[2H])C(F)(F)F)nc(N[C@]([2H])(C([2H])([2H])[2H])C(F)(F)F)n2)c1] | 6-(6-chloropyridin-2-yl-4-d)- N2,N4-bis((R)-1,1,1- trifluoropropan-2-yl-2,3,3,3-d4)- 1,3,5-triazine-2,4-diamine |
| 55 | 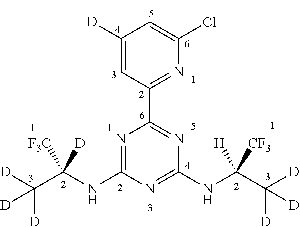 [SMILES: [2H]c1cc(Cl)nc(-c2nc(N[C@H](C([2H])([2H])[2H])C(F)(F)F)nc(N[C@]([2H])(C([2H])([2H])[2H])C(F)(F)F)n2)c1] | 6-(6-chloropyridin-2-yl-4-d)-N2- ((R)-1,1,1-trifluoropropan-2-yl- 2,3,3,3-d4)-N4-((R)-1,1,1- trifluoropropan-2-yl-3,3,3-d3)- 1,3,5-triazine-2,4-diamine |
| 56 | 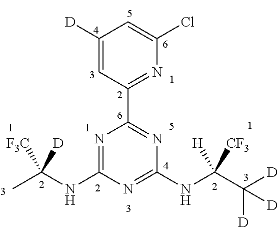 [SMILES: [2H]c1cc(Cl)nc(-c2nc(N[C@H](C([2H])([2H])[2H])C(F)(F)F)nc(N[C@]([2H])(C)C(F)(F)F)n2)c1] | 6-(6-chloropyridin-2-yl-4-d)-N2- ((R)-1,1,1-trifluoropropan-2-yl-2- d)-N4-((R)-1,1,1-trifluoropropan- 2-yl-3,3,3-d3)-1,3,5-triazine-2,4- diamine |
| 57 | 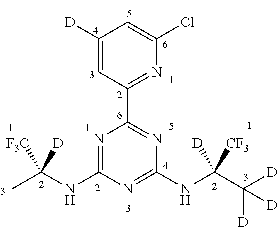 [SMILES: [2H]c1cc(Cl)nc(-c2nc(N[C@]([2H])(C([2H])([2H])[2H])C(F)(F)F)nc(N[C@]([2H])(C)C(F)(F)F)n2)c1] | 6-(6-chloropyridin-2-yl-4-d)-N2- ((R)-1,1,1-trifluoropropan-2-yl- 2,3,3,3-d4)-N44(R)-1,1,1- trifluoropropan-2-yl-2-d)-1,3,5- triazine-2,4-diamine |
| 58 | 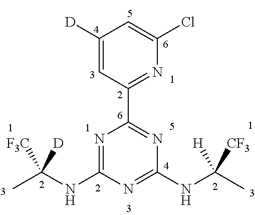 [SMILES: [2H]c1cc(Cl)nc(-c2nc(N[C@H](C)C(F)(F)F)nc(N[C@]([2H])(C)C(F)(F)F)n2)c1] | 6-(6-chloropyridin-2-yl-4-d)-N2- ((R)-1,1,1-trifluoropropan-2-yl)- N4-((R)-1,1,1-trifluoropropan-2-yl- 2-d)-1,3,5-triazine-2,4-diamine |
| 59 | 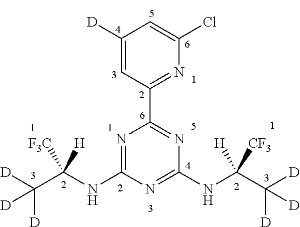 [SMILES: [2H]c1cc(Cl)nc(-c2nc(N[C@H](C([2H])([2H])[2H])C(F)(F)F)nc(N[C@H](C([2H])([2H])[2H])C(F)(F)F)n2)c1] | 6-(6-chloropyridin-2-yl-4-d)- N2,N4-bis((R)-1,1,1- trifluoropropan-2-yl-3,3,3-d3)- 1,3,5-triazine-2,4-diamine |
| 60 | 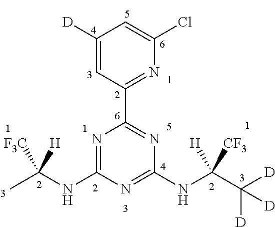 [SMILES: [2H]c1cc(Cl)nc(-c2nc(N[C@H](C([2H])([2H])[2H])C(F)(F)F)nc(N[C@H](C)C(F)(F)F)n2)c1] | 6-(6-chloropyridin-2-yl-4-d)-N2- ((R)-1,1,1-trifluoropropan-2-yl)- N4-((R)-1,1,1-trifluoropropan-2-yl- 3,3,3-d3)-1,3,5-triazine-2,4- diamine |
| 61 | 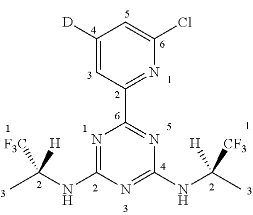 [SMILES: [2H]c1cc(Cl)nc(-c2nc(N[C@H](C)C(F)(F)F)nc(N[C@H](C)C(F)(F)F)n2)c1] | 6-(6-chloropyridin-2-yl-4-d)- N2,N4-bis((R)-1,1,1- trifluoropropan-2-yl)-1,3,5- triazine-2,4-diamine |
| 62 | 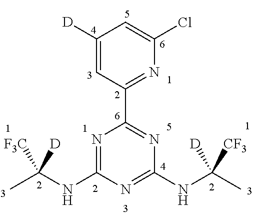 [SMILES: [2H]c1cc(Cl)nc(-c2nc(N[C@]([2H])(C)C(F)(F)F)nc(N[C@]([2H])(C)C(F)(F)F)n2)c1] | 6-(6-chloropyridin-2-yl-4-d)- N2,N4-bis((R)-1,1,1- trif1uoropropan-2-yl-2-d)-1,3,5- triazine-2,4-diamine |
| 63 | 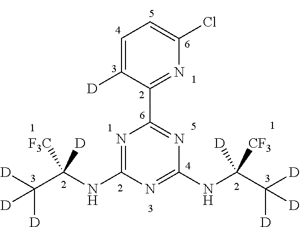 [SMILES: [2H]c1ccc(Cl)nc1-c1nc(N[C@]([2H])(C([2H])([2H])[2H])C(F)(F)F)nc(N[C@]([2H])(C([2H])([2H])[2H])C(F)(F)F)n1] | 6-(6-chloropyridin-2-yl-3-d)- N2,N4-bis((R)-1,1,1- trifluoropropan-2-yl-2,3,3,3-d4)- 1,3,5-triazine-2,4-diamine |
| 64 | 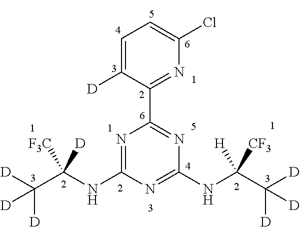 [SMILES: [2H]c1ccc(Cl)nc1-c1nc(N[C@H](C([2H])([2H])[2H])C(F)(F)F)nc(N[C@]([2H])(C([2H])([2H])[2H])C(F)(F)F)n1] | 6-(6-chloropyridin-2-yl-3-d)-N2- ((R)-1,1,1-trifluoropropan-2-yl- 2,3,3,3-d4)-N4-((R)-1,1,1- trifluoropropan-2-yl-3,3,3-d3)- 1,3,5-triazine-2,4-diamine |
| 65 | 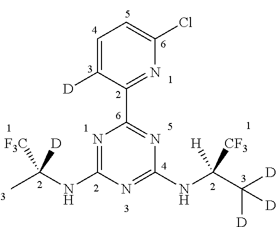 [SMILES: [2H]c1ccc(Cl)nc1-c1nc(N[C@H](C([2H])([2H])[2H])C(F)(F)F)nc(N[C@]([2H])(C)C(F)(F)F)n1] | 6-(6-chloropyridin-2-yl-3-d)-N2- ((R)-1,1,1-trifluoropropan-2-yl-2- d)-N4-((R)-1,1,1-trifluoropropan- 2-yl-3,3,3-d3)-1,3,5-triazine-2,4- diamine |
| 66 | 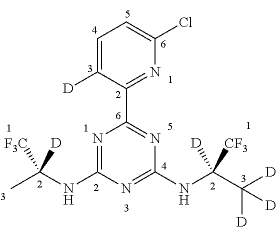 [SMILES: [2H]c1ccc(Cl)nc1-c1nc(N[C@]([2H])(C([2H])([2H])[2H])C(F)(F)F)nc(N[C@]([2H])(C)C(F)(F)F)n1] | 6-(6-chloropyridin-2-yl-3-d)-N2- ((R)-1,1,1-trifluoropropan-2-yl- 2,3,3,3-d4)-N4-((R)-1,1,1- trifluoropropan-2-yl-2-d)-1,3,5- triazine-2,4-diamine |
| 67 | 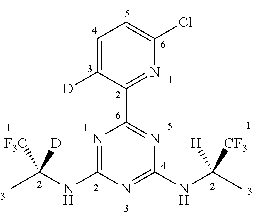 [SMILES: [2H]c1ccc(Cl)nc1-c1nc(N[C@H](C)C(F)(F)F)nc(N[C@]([2H])(C)C(F)(F)F)n1] | 6-(6-chloropyridin-2-yl-3-d)-N2- ((R)-1,1,1-trifluoropropan-2-yl)- N4-((R)-1,1,1-trifluoropropan-2-yl- 2-d)-1,3,5-triazine-2,4-diamine |
| 68 | 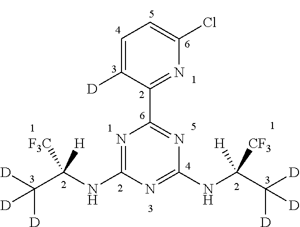 [SMILES: [2H]c1ccc(Cl)nc1-c1nc(N[C@H](C([2H])([2H])[2H])C(F)(F)F)nc(N[C@H](C([2H])([2H])[2H])C(F)(F)F)n1] | 6-(6-chloropyridin-2-yl-3-d)- N2,N4-bis((R)-1,1,1- trifluoropropan-2-yl-3,3,3-d3)- 1,3,5-triazine-2,4-diamine |
| 69 | 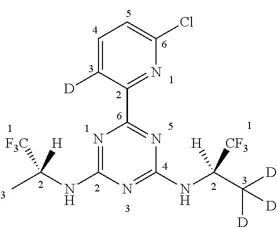 [SMILES: [2H]c1ccc(Cl)nc1-c1nc(N[C@H](C([2H])([2H])[2H])C(F)(F)F)nc(N[C@H](C)C(F)(F)F)n1] | 6-(6-chloropyridin-2-yl-3-d)-N2- ((R)-1,1,1-trifluoropropan-2-yl)- N4-((R)-1,1,1-trifluoropropan-2-yl- 3,3,3-d3)-1,3,5-triazine-2,4- diamine |
| 70 | 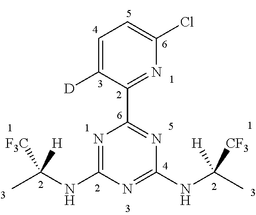 [SMILES: [2H]c1ccc(Cl)nc1-c1nc(N[C@H](C)C(F)(F)F)nc(N[C@H](C)C(F)(F)F)n1] | 6-(6-chloropyridin-2-yl-3-d)-N2- N2,N4-bis((R)-1,1,1- trifluoropropan-2-yl)-1,3,5- triazine-2,4-diamine |
| 71 | 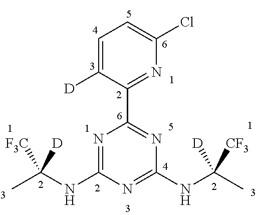 [SMILES: [2H]c1ccc(Cl)nc1-c1nc(N[C@]([2H])(C)C(F)(F)F)nc(N[C@]([2H])(C)C(F)(F)F)n1] | 6-(6-chloropyridin-2-yl-3-d)- N2,N4-bis((R)-1,1,1- trifluoropropan-2-yl-2-d)-1,3,5- triazine-2,4-diamine |
| 72 | 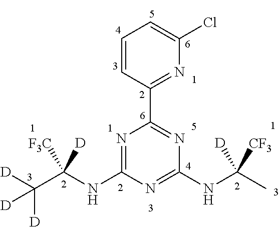 [SMILES: [2H]C([2H])([2H])[C@@]([2H])(Nc1nc(N[C@]([2H])(C)C(F)(F)F)nc(-c2cccc(Cl)n2)n1)C(F)(F)F] | 6-(6-chloropyridin-2-yl)-N2-((R)- 1,1,1-trifluoropropan-2-yl-2,3,3,3- d4)-N4-((R)-1,1,1-trifluoropropan- 2-yl-2-d)-1,3,5-triazine-2,4- diamine |
| 73 | 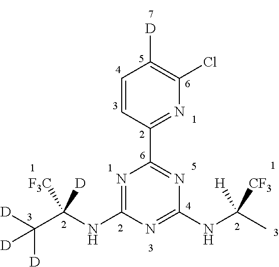 [SMILES: [2H]c1ccc(-c2nc(N[C@H](C)C(F)(F)F)nc(N[C@]([2H])(C([2H])([2H])[2H])C(F)(F)F)n2)nc1Cl] | 6-(6-chloropyridin-2-yl-5-d)-N2- ((R)-1,1,1-trifluoropropan-2-yl)- N4-((R)-1,1,1-trifluoropropan-2-yl- 2,3,3,3-d4)-1,3,5-triazine-2,4- diamine |
| 74 | 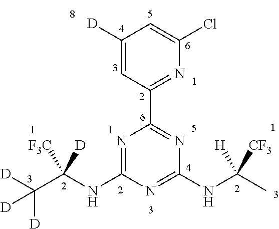 [SMILES: [2H]c1cc(Cl)nc(-c2nc(N[C@H](C)C(F)(F)F)nc(N[C@]([2H])(C([2H])([2H])[2H])C(F)(F)F)n2)c1] | 6-(6-chloropyridin-2-yl-4-d)-N2- ((R)-1,1,1-trifluoropropan-2-yl)- N4-((R)-1,1,1-trifluoropropan-2-yl- 2,3,3,3-d4)-1,3,5-triazine-2,4- diamine |
| 75 | 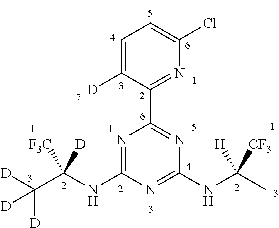 [SMILES: [2H]c1ccc(Cl)nc1-c1nc(N[C@H](C)C(F)(F)F)nc(N[C@]([2H])(C([2H])([2H])[2H])C(F)(F)F)n1] | 6-(6-chloropyridin-2-yl-3-d)-N2- ((R)-1,1,1-trifluoropropan-2-yl)- N4-((R)-1,1,1-trifluoropropan-2-yl- 2,3,3,3-d4)-1,3,5-triazine-2,4- diamine |
| 76 | 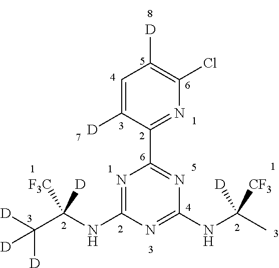 [SMILES: [2H]c1cc([2H])c(-c2nc(N[C@]([2H])(C([2H])([2H])[2H])C(F)(F)F)nc(N[C@]([2H])(C)C(F)(F)F)n2)nc1Cl] | 6-(6-chloropyridin-2-yl-3,5-d2)- N2-((R)-1,1,1-trifluoropropan-2-yl- 2,3,3,3-d4)-N4-((R)-1,1,1- trifluoropropan-2-yl-2-d)-1,3,5- triazine-2,4-diamine |
| 77 | 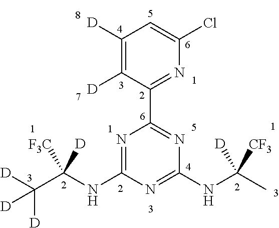 [SMILES: [2H]c1cc(Cl)nc(-c2nc(N[C@]([2H])(C([2H])([2H])[2H])C(F)(F)F)nc(N[C@]([2H])(C)C(F)(F)F)n2)c1[2H]] | 6-(6-chloropyridin-2-yl-3,4-d2)- N2-((R)-1,1,1-trifluoropropan-2-yl- 2,3,3,3-d4)-N4-((R)-1,1,1- trifluoropropan-2-yl-2-d)-1,3,5- triazine-2,4-diamine |
| 78 | 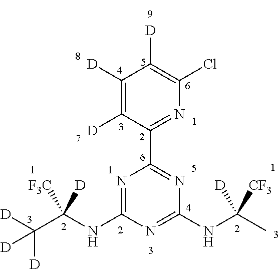 [SMILES: [2H]c1c(Cl)nc(-c2nc(N[C@]([2H])(C([2H])([2H])[2H])C(F)(F)F)nc(N[C@]([2H])(C)C(F)(F)F)n2)c([2H])c1[2H]] | 6-(6-chloropyridin-2-yl-3,4,5-d3)- N2-((R)-1,1,1-trifluoropropan-2-yl- 2,3,3,3-d4)-N4-((R)-1,1,1- trifluoropropan-2-yl-2-d)-1,3,5- triazine-2,4-diamine |
| 79 | 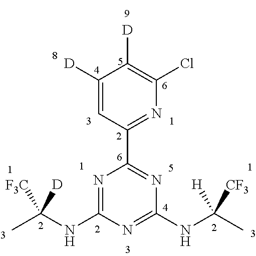 [SMILES: [2H]c1cc(-c2nc(N[C@H](C)C(F)(F)F)nc(N[C@]([2H])(C)C(F)(F)F)n2)nc(Cl)c1[2H]] | 6-(6-chloropyridin-2-yl-4,5-d2)- N2,N4-bis((R)-1,1,1- trifluoropropan-2-yl)-1,3,5- triazine-2,4-diamine |

Brain Tumors Treated by Methods of the Application

The methods of the application are useful for treating brain tumors characterized by the presence of an IDH1 and/or IDH2 mutation. This includes all tumors characterized by the presence of an IDH1 and/or IDH2 mutation inside the human skull (cranium) or in the central spinal canal. The tumor may originate from the brain itself, but also from lymphatic tissue, blood vessels, the cranial nerves, the brain envelopes (meninges), skull, pituitary gland, or pineal gland. Within the brain itself, the involved cells may be neurons or glial cells (which include astrocytes, oligodendrocytes, and ependymal cells). Brain tumors may also spread from cancers primarily located in other organs (metastatic tumors).

In some embodiments, the brain tumor characterized by the presence of an IDH1 and/or IDH2 mutation is a glioma, such as an ependymoma, astrocytoma, oligoastrocytoma, oligodendroglioma, ganglioglioma, glioblastoma (also known as glioblastoma multiforme), or mixed glioma. Gliomas are primary brain tumors and are classified into four grades (I, II, III, and IV) based on their appearance under a microscope, and particularly the presence of atypical cells, mitoses, endothelial proliferation, and necrosis. Grade I and II tumors, termed “low-grade gliomas,” have none or one of these features and include diffuse astrocytomas, pilocytic astrocytomas, low-grade astrocytomas, low-grade oligoastrocytomas, low-grade oligodendrogliomas, gangliogliomas, dysembryoplastic neuroepithelial tumors, pleomorphic xanthoastrocytomas, and mixed gliomas. Grade III and IV tumors, termed “high-grade gliomas,” have two or more of these features and include anaplastic astrocytomas, anaplastic oligodendrogliomas, anaplastic oligoastrocytomas, anaplastic ependymomas, and glioblastomas (including giant cell glioblastomas and gliosarcomas). In one aspect of these embodiments, the glioma is a low-grade glioma. In another aspect of these embodiments, the glioma is a high-grade glioma. In another aspect of these embodiments, the glioma is a glioblastoma.

In some embodiments, the brain tumor (e.g., glioma) to be treated is characterized by the presence of an IDH1 mutation, wherein the IDH1 mutation results in accumulation of R(−)-2-hydroxyglutarate in a patient. In one aspect of these embodiments, the IDH1 mutation results in accumulation of R(−)-2-hydroxyglutarate in a patient by providing a new ability of the enzyme to catalyze the NADPH-dependent reduction of α-ketoglutarate to R(−)-2-hydroxyglutarate in a patient. In another aspect of these embodiments, the IDH1 mutation is an R132X mutation. In another aspect of these embodiments, the R132X mutation is selected from R132H, R132C, R132L, R132V, R132S and R132G. In another aspect of these embodiments, the R132X mutation is R132H or R132C. In yet another aspect of these embodiments, the R132X mutation is R132H. In still another aspect of these embodiments, at least 30, 40, 50, 60, 70, 80 or 90% of the brain tumor (e.g., glioma) cells carry an IDH1 R132X mutation, such as an R132H, R132C, R132L, R132V, R132S or R132G mutation, at the time of diagnosis or treatment. A brain tumor (e.g., glioma) can be analyzed by sequencing cell samples to determine the presence and specific nature of (e.g., the changed amino acid present at) a mutation at amino acid 132 of IDH1.

In other embodiments, the brain tumor (e.g., glioma) to be treated is characterized by the presence of an IDH2 mutation, wherein the IDH2 mutation results in accumulation of R(−)-2-hydroxyglutarate in a patient. In one aspect of these embodiments, the IDH2 mutation results in accumulation of R(−)-2-hydroxyglutarate in a patient by providing a new ability of the enzyme to catalyze the NADPH-dependent reduction of α-ketoglutarate to R(−)-2-hydroxyglutarate in a patient. In another aspect of these embodiments, the mutant IDH2 has an R140X mutation. In another aspect of these embodiments, the R140X mutation is a R140Q mutation. In another aspect of these embodiments, the R140X mutation is a R140W mutation. In another aspect of these embodiments, the R140X mutation is a R140L mutation. In another aspect of these embodiments, the mutant IDH2 has an R172X mutation. In another aspect of these embodiments, the R172X mutation is a R172K mutation. In another aspect of these embodiments, the R172X mutation is a R172G mutation. In still another aspect of these embodiments, at least 30, 40, 50, 60, 70, 80 or 90% of the brain tumor (e.g., glioma) cells carry an IDH2 R140X and/or R172X mutation, such as an R140Q, R140W, or R140L and/or R172K or R172G mutation, at the time of diagnosis or treatment. A brain tumor (e.g., glioma) can be analyzed by sequencing cell samples to determine the presence and specific nature of (e.g., the changed amino acid present at) a mutation at amino acid 140 and/or 172 of IDH2.

In still other embodiments, the brain tumor (e.g., glioma) to be treated is characterized by the presence of an IDH1 mutation and an IDH2 mutation, wherein the IDH1 and IDH2 mutations collectively result in accumulation of R(−)-2-hydroxyglutarate in a patient. In one aspect of these embodiments, the IDH1 and IDH2 mutations result in accumulation of R(−)-2-hydroxyglutarate in a patient by providing a new ability of the enzyme to catalyze the NADPH-dependent reduction of α-ketoglutarate to R(−)-2-hydroxyglutarate in a patient. In various aspects of these embodiments, the IDH1 mutation is an R132X mutation selected from R132H, R132C, R132L, R132V, R132S and R132G. In various aspects of these embodiments, the IDH2 mutation is an R140Q, R140W, R140L, R172K or R172G mutation. In various other aspects of these embodiments, the brain tumor (e.g., glioma) to be treated is characterized by any combination of the foregoing IDH1 and IDH2 mutations. In still other aspects of these embodiments, at least 30, 40, 50, 60, 70, 80 or 90% of the brain tumor (e.g., glioma) cells carry an IDH1 R132X mutation, such as an R132H, R132C, R132L, R132V, R132S or R132G mutation, and an IDH2 R140X and/or R172X mutation, such as an R140Q, R140W, or R140L and/or R172K or R172G mutation, at the time of diagnosis or treatment. A brain tumor (e.g., glioma) can be analyzed by sequencing cell samples to determine the presence and specific nature of (e.g., the changed amino acid present at) a mutation at amino acid 132 of IDH1 and at amino acid 140 and/or 172 of IDH2.

In still other embodiments, the brain tumor (e.g., glioma) to be treated is characterized by the presence of an IDH1 allele that does not include an R132X mutation and an IDH2 allele that does not include an R140X or R172X mutation. In one aspect of these embodiments, at least 90% of the brain tumor (e.g., glioma) cells do not include a mutation at amino acid 132 of IDH1 or at amino acid 140 or 172 of IDH2 at the time of diagnosis or treatment. A brain tumor (e.g., glioma) can be analyzed by sequencing cell samples to determine the presence or absence of a mutation at amino acid 132 of IDH1 and at amino acid 140 and/or 172 of IDH2.

Compounds Used in Methods of the Application

Compounds of formula (I) (including all of the embodiments described herein), or pharmaceutically acceptable salts or pharmaceutically acceptable co-crystalline materials thereof, are used in the methods described herein.

Compositions and Routes of Administration

Compounds of formula (I) (including all of the embodiments described herein), or pharmaceutically acceptable salts or pharmaceutically acceptable co-crystalline materials thereof, may be formulated together with a pharmaceutically acceptable carrier, adjuvant, or vehicle into pharmaceutical compositions prior to being administered to a subject.

The term “pharmaceutically acceptable carrier, adjuvant, or vehicle” refers to a carrier, adjuvant, or vehicle that may be administered to a subject, together with a compound of formula (I), and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of a compound of formula (I).

The pharmaceutical compositions may be administered orally, parenterally, by inhalation spray, topically, rectally, nasally, buccally, vaginally or via an implanted reservoir, preferably by oral administration or administration by injection. The pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions may also be administered in the form of suppositories for rectal administration. These compositions can be prepared by mixing a compound of formula (I) with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions may be administered topically to the skin. The pharmaceutical composition should be formulated with a suitable ointment containing the active components suspended or dissolved in a carrier. Carriers for topical administration of the compounds of one aspect of this application include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of one aspect of this application may also be topically applied to the lower intestinal tract by rectal suppository formulation or in a suitable enema formulation. Topically-transdermal patches are also included in one aspect of this application.

The pharmaceutical compositions may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The amount of active ingredient that may be combined with the carrier materials to produce a single dosage form will vary depending upon the patient treated and the particular mode of administration. A typical preparation will contain from about 5% to about 95% active compound (w/w). Alternatively, such preparations contain from about 20% to about 80% active compound.

The pharmaceutical compositions comprising the compound of formula (I) may further comprise another therapeutic agent useful for treating cancer, such as a DNA-reactive agent (defined above).

Radiation Therapy

Radiation therapy involves the use of high-energy radiation (e.g., x-rays, gamma rays, or charged particles) to damage and/or kill cancer cells and to shrink tumors. In the methods of the application, radiation may be delivered to the brain tumor (e.g., glioma) by a machine positioned outside the body (external-beam radiation therapy), by radioactive material placed in the body near the brain tumor (internal radiation therapy, also called brachytherapy), or by radioactive substances administered systemically (e.g., radioactive iodine) that travel through the bloodstream to the brain tumor. Alternatively, these delivery methods can be used in combination.

In some embodiments, the radiation therapy comprises external radiation therapy (e.g., external-beam radiation therapy including fractionated external-beam radiation therapy, stereotactic radiation such as Cyberknife® or Gamma Knife®, proton therapy, and the like), where the radiation is delivered to the brain tumor (e.g., glioma) by an instrument outside the body. External radiation therapy may be given as a course of several treatments over days or weeks. In one aspect of these embodiments, the radiation is administered in the form of x-rays.

In other embodiments, the radiation therapy comprises internal radiation therapy, where the radiation comes from an implant or a material (liquid, solid, semi-solid or other substance) placed inside the body. In one aspect of these embodiments, the internal radiation therapy is brachytherapy, where a solid radioactive source is placed inside the body near the brain tumor. In another aspect of these embodiments, the internal radiation therapy comprises the systemic administration of a radiation source, typically a radionuclide (radioisotope or unsealed source). The radiation source may be orally administered or may be injected into a vein.

Additional Treatments and Therapeutic Agents

In some embodiments, the methods described herein further comprise the additional step of administering to the patient an additional cancer therapeutic agent or an additional cancer treatment.

For example, the methods described herein may be practiced in combination with the existing standard of care therapy for glioma. The standard of care for patients diagnosed with glioma considers the tumor location, potential symptoms, and potential benefits versus risks of the different treatment options (modalities). Upon initial diagnosis of glioma, standard treatment consists of maximal surgical resection, radiotherapy, and/or concomitant and adjuvant chemotherapy (e.g. with temozolomide (TMZ)). For patients older than 70 years, less aggressive therapy is sometimes employed, using radiation TMZ alone. (See generally National Comprehensive Cancer Network Guidelines, version 1.2016 available at nccn.org.)

For example, the current regimen for treatment of primary grade IV glioblastoma (GBM) is surgical resection in combination with radiation therapy and chemotherapy. Current U.S. FDA approved chemotherapies for primary grade IV GBM tumors include nitrosoureas (lomustine and carmustine) and TMZ. Glioma post-surgical standard of care therapy consists of radiation and TMZ as antineoplastic therapy and dexamethasone (DEX) for neurological symptomatic relief. More recently, the antibody to vascular endothelial growth factor (VEGF), bevacizumab, is being used more often for tumor recurrence. Numerous experimental agents are in various phases of pre-clinical and clinical application are in development and may result in changes to the standard of care for glioblastoma.

The methods described herein can be combined with radiation therapy or surgery. In certain embodiments, the methods are practiced on a patient who is undergoing radiation therapy, has previously undergone radiation therapy or will be undergoing radiation therapy. In certain embodiments, the methods are practiced on a patient who has undergone brain tumor removal surgery. Further provided herein are methods for treating patients who have been previously treated for a brain tumor, but are non-responsive to standard therapies, for example with Temozolomide, as well as those who have not previously been treated. Further provided herein are methods for treating patients who have undergone surgery in an attempt to treat the condition at issue, as well as those who have not. Because patients with brain tumors may have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with a brain tumor. In some embodiments, the methods described herein additionally comprise administration of Temozolomide. In some such embodiments, the brain tumor is Temozolomide resistant.

Exemplary additional cancer therapeutic agents include for example, chemotherapy, targeted therapy, immunotherapy, anti-epileptics, steroids, CAR-Ts, Gliadel® (carmustine implant), and Avastin® (bevacizumab). Additional cancer treatments include, for example: surgery, and radiation therapy.

In some embodiments the additional cancer therapeutic agent is a targeted therapy agent. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors such as Axitinib, Bosutinib, Cediranib, dasatinib, erlotinib, imatinib, gefitinib, lapatinib, Lestaurtinib, Nilotinib, Semaxanib, Sorafenib, Sunitinib, and Vandetanib, and also cyclin-dependent kinase inhibitors such as Alvocidib and Seliciclib. In some embodiments, the targeted therapy can be used in combination with the methods described herein, e.g., a biguanide such as metformin or phenformin, preferably phenformin.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decays in the vicinity of the cell. An example of such therapy includes BEXXAR®.

In some embodiments, the additional cancer therapeutic agent is an immunotherapy agent. Cancer immunotherapy refers to a diverse set of therapeutic strategies designed to induce the subject's own immune system to fight the tumor.

Allogeneic hematopoietic stem cell transplantation can be considered a form of immunotherapy, since the donor's immune cells will often attack the tumor in a graft-versus-tumor effect. In some embodiments, the immunotherapy agents can be used in combination with the methods described herein.

Other possible additional therapeutic modalities include imatinib, gene therapy, peptide and dendritic cell vaccines, synthetic chlorotoxins, and radiolabeled drugs and antibodies.

### EXAMPLES

Compounds of formula (I), or pharmaceutically acceptable salts or pharmaceutically acceptable co-crystalline materials thereof, may be evaluated for potential efficacy against human neurosphere-derived grade III glioma cells carrying an IDH1 R132H mutation:

(1) alone, as a monotherapy;

(2) in combination with focal beam radiation; and/or

(3) in combination with an additional therapeutic agent, such as temozolomide; via methods analogous to those described in WIPO Publication No. 2018/231796 A1, which is incorporated herein by reference. The additional therapeutic agents are described in greater detail in the following examples.

### Abbreviations

Unless otherwise noted, or where the context dictates otherwise, the following abbreviations shall be understood to have the following meanings:

| Abbreviation | Meaning |
|---|---|
| IDH1 | Isocitrate Dehydrogenase 1 |
| IDH1m | Mutant Isocitrate Dehydrogenase 1 |
| R132H | Arginine to histidine point mutation at codon |
| | 132 of IDH1 |
| IDH1^{R132H} | IDH1 having an R132H point mutation |
| EGF | Epidermal growth factor |
| bFGF | Basic fibroblast growth factor |
| MRI | Magnetic resonance imaging |
| 2HG | 2-hydroxyglutarate |
| PO | Per Os (oral administration) |
| SARRP | Small Animal Radiation Research Platform |
| QD | Quaque Die (administration once per day) |
| Q12H | Administration every 12 hours |
| Q12Hx2 | Administration every 12 hours for 2 |
| | administrations |
| (Q12Hx2) QDx17 | Administration every 12 hours for 2 |
| | administrations per day for 17 days (34 total |
| | administrations), could also be written as |
| | “Q12Hx34” or “every 12 hours for 17 days” |
| BID | Bis in Die (administration twice per day) |
| T2w | T2-weighted |
| rcf | Relative centrifugal force |
| TMZ | Temozolomide |
| Gy | Gray |
| RT | Radiation therapy |
| BED | Biological effective dose |
| mm | Millimeters |
| mg | Milligrams |
| ng | Nanograms |
| kg | kilograms |
| mL | Milliliters |
| min | Minutes |
| MAD | Median absolute distribution |
| SEM | Standard error of the mean |

### Example 1

### Combination of a Compound of Formula (I) and Radiation Therapy in IDH1m Glioma Model Study Objective:

The objective of this study is to evaluate the potential efficacy of a compound of formula (I), or a pharmaceutically acceptable salt or a pharmaceutically acceptable co-crystalline material thereof, given twice daily, alone and in combination with focal beam radiation, against established orthotopic human neurosphere-derived grade Ill glioma cells carrying an IDH1 R132H mutation in female mice using magnetic resonance imaging (MRI).

### Study Design:

The study mice are imaged by MRI on Days 37 and 38 post inoculation and sorted into five study groups based on MRI estimation of tumor burden. Staging values are recorded on Day 38. Treatment begins on Day 39 post inoculation with the treatment schedules summarized in Table 1.

TABLE 1
Study Design/Treatment Schedules

| | # of | | | |
|---|---|---|---|---|
| Group | Animals | Treatment | Route | Dose and Schedule |
| 1 | 10 | Vehicle Control | PO | 0.2 mL/20 g, (Q12Hx2) QDx17 |
| | | (0.5% | | (Days 39-59) |
| | | methylcellulose/ | | |
| | | 0.2% Tween80 in | | |
| | | water) | | |
| 2 | 10 | Focal Radiation | SARRP | Study Animals 1-5: 2 Gy, QDx5 |
| | | (SARRP) | | (Days 39-43) |
| | | | | Study Animals 6-10: 2 Gy, QDx5 |
| | | | | (Days 39-41 and 44-45) |
| 3 | 10 | compound of | PO | 50 mg/kg, (Q12Hx2) QDx17 |
| | | formula (I) | | (Days 39-63)* |
| 4 | 10 | compound of | PO + | compound of formula (I): 50 mg/kg, |
| | | formula (I) + Focal | SARRP | (Q12Hx2) QDx17 |
| | | Radiation (SARRP) | | (Days 39-77) |
| | | (simultaneous | | Focal Radiation: 2 Gy, QDx5 |
| | | treatment) | | (Days 39-41 and 44-45)** |
| 5 | 10 | Focal Radiation | SARRP, | Focal Radiation: 2 Gy, QDx5 |
| | | (SARRP), then | then PO | (Days 39-41 and 44-45) |
| | | compound of | | compound of formula (I): 50 mg/kg, |
| | | formula (I) | | (Q12Hx2) QDx17 |
| | | (sequential | | (Days 46-75) |
| | | treatment) | | |

### Materials and Methods:

The study animals are implanted intracranially on Day 0 of the study with 5×10⁴ cells bearing the IDH1^{R132H} mutation. The tumors are staged for enrollment on Day 38 at a small tumor volume (mean 9.1 mm³).

A compound of formula (I), or a pharmaceutically acceptable salt or a pharmaceutically acceptable co-crystalline material thereof, is prepared to meet dose level requirements. The compound is formulated at a concentration of 5 mg/mL in a vehicle of 0.5% methylcellulose, 0.2% Tween 80, and water. A polytron is used for approximately 30-60 seconds to dissolve any clumps. The resulting formulation is a fine, white suspension with a pH value of 2.8. The formulation is prepared fresh daily, and is stirred for at least one hour prior to dosing. The dosing formulation is stored at 4° C. between doses.

The compound of formula (I) is orally dosed at 50 mg/kg (based on the amount of compound of formula (I)), twice daily, for Groups 3-5. The dose of the compound of formula (I) is chosen based on historical data that at this dose, 2HG production is inhibited at >98% within the brain tumors, when compared to healthy brain tissue.

Radiation treatment is administered via the Xstrahl Life Sciences Small Animal Radiation Research Platform, or SARRP. This system is designed to allow for highly targeted irradiation, which mimics that applied in human patients. The x-ray tube on the SARRP has variable output and is used for Computed Tomography (CT) imaging to guide treatment and also for treatment delivery with single or multiple beams. The total amount of radiation delivered to the tumor is 10 Gy/mouse (2 Gy, QD×5) for Groups 2, 4, and 5.

Group 1 is anesthetized on the same treatment schedule.

All of the study animals begin to receive subcutaneous fluids (lactated ringers) on Day 44. Hydrogel supplement is added to all cages beginning on Day 39.

T2-weighted (T2w) magnetic resonance images (MRI) are acquired such that volumetric measurements could be assessed to determine disease progression.

Brain tumor volumes are evaluated via MRI on Days 38, 45, 49, 52, 56, 59, 63, 66, and 71.

### Results:

The results of these experiments will be evaluated to determine whether the combination of a compound of formula (I) and radiation therapy shows antagonism in vivo in an orthotopic mutant IDH1 glioma brain tumor model.

### Example 2

### Combination of a Compound of Formula (I) and Temozolomide Therapy in IDH1m Glioma Model

### Study Objective:

The objective of this study is to evaluate the potential efficacy of a compound of formula (I), or a pharmaceutically acceptable salt or a pharmaceutically acceptable co-crystalline material thereof, given twice daily, alone and in combination with temozolomide against established subcutaneous human neurosphere-derived glioma cells carrying an IDH1 R132H mutation, in male mice.

### Study Design:

The study mice are divided into six study groups, which are treated in accordance with the treatment schedules summarized in Table 4.

TABLE 4
Study Design

| | # of | | |
|---|---|---|---|
| Group | Animals | Treatment | Dose and Schedule |
| 1 | 10 | Vehicle Control | 5 mL/kg, BID |
| 2 | 10 | TMZ | 10 mg/kg (5 mL/kg), |
| | | | Monday-Thursday |
| 3 | 10 | Compound of | 50 mg/kg (5 mL/kg), BID |
| | | formula (I) | |
| 4 | 10 | Compound of | 2 mg/kg (5 mL/kg), BID |
| | | formula (I) | |
| | | TMZ | 10 mg/kg (5 mL/kg), |
| | | | Monday-Thursday |
| 5 | 10 | Compound of | 10 mg/kg (5 mL/kg), BID |
| | | formula (I) | |
| | | TMZ | 10 mg/kg (5 mL/kg), |
| | | | Monday-Thursday |
| 6 | 10 | Compound of | 50 mg/kg (5 mL/kg), BID |
| | | formula (I) | |
| | | TMZ | 10 mg/kg (5 mL/kg), |
| | | | Monday-Thursday |

### Materials and Methods

About ninety (90) 5-6 week old male mice are implanted subcutaneously with 1×10⁶ cells bearing the IDH1^{R132H} mutation in growth hormone/heparin free media with Matrigel (Final, 1:1). Excess mice are inoculated to account for tumor variability. Tumor volume and body weights are monitored twice a week until tumors reached ˜200 mm³. Once tumors reached ˜200 mm³ animals are randomized into 6 groups based on digital caliper estimation of tumor burden.

The Vehicle Control formulation (Group 1) contains 0.5% methylcellulose and 0.1% Tween 80 in water, and is adjusted with hydrochloric acid to pH 3.5.

The TMZ (2 mg/mL) formulation (Groups 2, 4, 5, and 6) is prepared as follows:

1) 24 mg of TMZ is weighed into a clear vial.

2) 12 ml of 0.5% methylcellulose and 0.1% Tween 80 in water is added.

3) The vial is vortexed for 1-2 min, sonicated if needed, and stored on ice prior to use.

The compound of formula (I) (10 mg/mL) formulation (Groups 3 and 6) is prepared as follows:

1) 120 mg of a compound of formula (I) is weighed into a clear vial.

2) 12 ml of 0.5% methylcellulose and 0.1% Tween 80 in water is added.

3) The vial is vortexed for 1-2 min, sonicated if needed, and stored on ice prior to use.

The Compound of formula (I) (2 mg/mL) formulation (Group 5) is prepared as follows:

1) 1.4 mL of the 10 mg/mL formulation is transferred into a clear vial.

2) 5.6 mL of 0.5% methylcellulose and 0.1% Tween 80 in water is added.

3) The vial is vortexed for 1-2 min, sonicated if needed, and stored on ice prior to use.

The Compound of formula (I) (0.4 mg/mL) formulation (Group 4) is prepared as follows:

1) 1.2 mL of the 2 mg/mL formulation is transferred into a clear vial.

2) 4.8 mL of 0.5% methylcellulose and 0.1% Tween 80 in water is added.

3) The vial is vortexed for 1-2 min, sonicated if needed, and stored on ice prior to use.

As indicated in Table 3, each formulation is administered via oral gavage at 5 ml/kg, based on the most recent body weight. The Vehicle Control and the compound of formula (I) formulations are administered BID, 7 days/week, beginning on Day 1 after randomization of the animals into groups. The TMZ formulation is administered once per day on Monday-Thursday, followed by 3 days off beginning on Day 1 after randomization of the animals into groups. For administration of TMZ, animals are dosed at least 1 hour apart from administration of the compound of formula (I) to allow mice to recover from initial gavage dose. The study is conducted for 55 days.

### Results:

The results will be evaluated to determine whether the combination of a compound of formula (I) and TMZ results in any change in anti-tumor activity when compared to each monotherapy.

### Example 3

### Combination of a Compound of Formula (I) and Radiation Therapy in IDH1m Glioma Study Objective:

The objective of this study is to evaluate the potential efficacy of a compound of formula (I), or a pharmaceutically acceptable salt or a pharmaceutically acceptable co-crystalline material thereof, given twice daily, alone and in combination with focal beam radiation, against established orthotopic human neurosphere-derived grade III glioma cells carrying an IDH1 R132H mutation in female mice using survival as the end point.

### Study Design

The study mice are imaged by MRI on Days 37 and 38 post inoculation and sorted into five study groups based on MRI estimation of tumor burden. Staging values are recorded on Day 38. Treatment is begun on Day 40 post inoculation with the treatment and schedules are summarized in Table 5.

TABLE 5
Study Design/Treatment Schedules

| | # of | | | |
|---|---|---|---|---|
| Group | Animals | Treatment | Route | Dose and Schedule |
| 1 | 10 | Vehicle Control | PO | 0.2 mL/20 g, (Q12Hx2) QDx17 |
| | | (0.5% | | |
| | | methylcellulose/ | | |
| | | 0.2% Tween80 in | | |
| | | water) | | |
| 2 | 10 | Focal Radiation | SARRP | 2 Gy, 10 mm Collimator, QDx5 |
| | | (SARRP) | | |
| 3 | 10 | Compound of | PO | 50 mg/kg, (Q12Hx2) QDx17 |
| | | formula (I) | | |
| 4 | 10 | Compound of | PO + | Compound of formula (I): 50 mg/kg, |
| | | formula (I) + Focal | SARRP | (Q12Hx2) QDx17 |
| | | Radiation (SARRP) | | Focal Radiation: 2 Gy, 10 mm |
| | | (simultaneous | | Collimator, QDx5 |
| | | treatment) | | |
| 5 | 10 | Focal Radiation | SARRP, | Focal Radiation: 2 Gy, 10 mm |
| | | (SARRP), then | then PO | Collimator, QDx5 |
| | | Compound of | | Compound of formula (I): 50 mg/kg, |
| | | formula (I) | | (Q12Hx2) QDx17 |
| | | (sequential | | |
| | | treatment) | | |

### Materials and Methods:

The study animals are implanted intracranially on Day 0 of the study with 5×10⁴ cells bearing the IDH1^{R132H} mutation.

T2-weighted (T2w) magnetic resonance images (MRI) are acquired such that volumetric measurements can be assessed to determine disease progression.

All mice are sorted into study groups based on magnetic resonance estimation of tumor burden. The mice are distributed to ensure that the mean tumor burden for all groups is within 10% of the overall mean tumor burden for the study population. Treatment begins on Day 40. All mice are dosed according to individual body weight (0.2 mL/20 g) or at a fixed volume on the day of treatment.

Hydrogel® supplementation is added to all cages for all study mice at the start of the study (Day 40), and is replenished daily until study termination.

Radiation treatment is administered via the Xstrahl Life Sciences Small Animal Radiation Research Platform, or SARRP. This system has been designed to allow for highly targeted irradiation which mimics that applied in human patients. The x-ray tube on the SARRP has variable output and is used for Computed Tomography (CT) imaging to guide treatment and also for treatment delivery with single or multiple beams. The total amount of radiation delivered to the tumor is 10 Gy/mouse (2 Gy, QD×5) for Groups 2, 4, and 5.

At 6 hours after the morning dose of a compound of formula (I), mice that exceed euthanasia criteria (weight loss in excess of 30%, distended cranium, severely impaired movement, severe respiratory distress, and/or loss of righting reflex) are euthanized via overexposure to carbon dioxide for blood and brain collection.

### Measurement and Endpoints:

The primary endpoint used for efficacy is increased lifespan.

Assessment of Side Effects. All animals are observed for clinical signs at least once daily. Animals are weighed on each day of treatment.

Treatment related body weight loss and net treatment related body weight loss are also determined. Treatment related body weight loss is evaluated for consistency with disease progression and any relationship to treatments on study.

Median Lifespan. The lifespan of each animal is measured from the day of first treatment (not the day of tumor implant) for each animal (Kaplan-Meier Survival—Log-Rank) and is used to calculate the median lifespan for each group. The calculation is based on the day of death for all animals that either die or are euthanized for disease or treatment related causes. Animals euthanized for sampling or therapy unrelated causes are excluded from this calculation.

The median lifespan for each group is used to calculate the % increase in lifespan (% ILS). % ILS is a group endpoint. It is calculated as follows:

% ILS={[(median treated lifespan)−(median control lifespan)]/(median control lifespan)}*

P values and statistical significance for a comparison of the treatment groups (Groups 2-5) to the control group (Group 1) are determined using SigmaPlot 12.5 software.

Results:

The mean estimated tumor burden for all groups in the experiment is evaluated.

The median lifespans and % ILS of Groups 2-5 are evaluated.

What is claimed is:

1. A compound of formula (I) 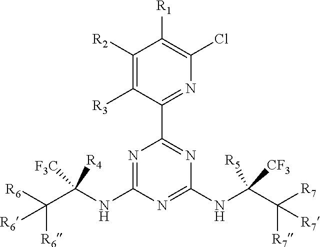
wherein each of R₁, R₂, R₃, R₄, R₅, R₆, R_{6′}, R_{6″}, R₇, R_{7′}, and R_{7″} is independently H or D, provided that at least one of R₁, R₂, R₃, R₄, R₅, R₆, R_{6′}, R_{6″}, R₇, R_{7′}, and R_{7″} is D, or a pharmaceutically acceptable salt or a pharmaceutically acceptable co-crystalline material thereof.

2. The compound of claim 1, or a pharmaceutically acceptable salt or a pharmaceutically acceptable co-crystalline material thereof, wherein any one, or two, or all three of R₁, R₂, and R₃ is D.

3. The compound of claim 2, or a pharmaceutically acceptable salt or a pharmaceutically acceptable co-crystalline material thereof, wherein (i) R₄ is D, and R₅ is H; or (ii) R₄ is H, and R₅ is D.

4. The compound of claim 2, or a pharmaceutically acceptable salt or a pharmaceutically acceptable co-crystalline material thereof, wherein each of R₄ and R₅ is D.

5. The compound of claim 2, or a pharmaceutically acceptable salt or a pharmaceutically acceptable co-crystalline material thereof, wherein at least one of R₆, R_{6′}, R_{6″}, R₇, R_{7′}, and R_{7″} is D.

6. The compound of claim 2, or a pharmaceutically acceptable salt or a pharmaceutically acceptable co-crystalline material thereof, wherein (i) each of R₆, R_{6′}, and R_{6″} is D; or (ii) each of R₇, R_{7′}, and R_{7″} is D.

7. The compound of claim 2, or a pharmaceutically acceptable salt or a pharmaceutically acceptable co-crystalline material thereof, wherein each of R₄, R₅, R₆, R_{6′}, R_{6″}, R₇, R_{7′}, and R_{7″} is D.

8. The compound of claim 1, or a pharmaceutically acceptable salt or a pharmaceutically acceptable co-crystalline material thereof, wherein (i) R₄ is D, and R₅ is H; or (ii) R₄ is H, and R₅ is D.

9. The compound of claim 1, or a pharmaceutically acceptable salt or a pharmaceutically acceptable co-crystalline material thereof, wherein each of R₄ and R₅ is D.

10. The compound of claim 1, or a pharmaceutically acceptable salt or a pharmaceutically acceptable co-crystalline material thereof, wherein at least one of R₆, R_{6′}, R_{6″}, R₇, R_{7′}, and R_{7″} is D.

11. The compound of claim 1, or a pharmaceutically acceptable salt or a pharmaceutically acceptable co-crystalline material thereof, wherein (i) each of R₆, R_{6′}, and R_{6″} is D; or (ii) each of R₇, R_{7′}, and R_{7″} is D.

12. The compound of claim 1, or a pharmaceutically acceptable salt or a pharmaceutically acceptable co-crystalline material thereof, wherein each of R₄, R₅, R₆, R_{6′}, R_{6″}, R₇, R_{7′}, and R_{7″} is D.

13. The compound of claim 1, or a pharmaceutically acceptable salt or a pharmaceutically acceptable co-crystalline material thereof, wherein each of R₁, R₂, R₃, R₄, R₅, R₆, R_{6′}, R_{6″}, R₇, R_{7′}, and R_{7″} is D.

14. The compound of claim 1 that is chosen from the compounds provided in Table 1, or a pharmaceutically acceptable salt or a pharmaceutically acceptable co-crystalline material thereof.

15. The co-crystalline material of claim 1 comprising a compound of formula I and citric acid.

16. A method for treating a brain tumor characterized by the presence of an IDH1 and/or IDH2 mutation in a patient in need thereof comprising administering to the patient a compound of claim 1, or a pharmaceutically acceptable salt or a pharmaceutically acceptable co-crystalline material thereof, in amounts effective for treating the brain tumor.

17. The method of claim 16 further comprising radiation therapy in an amount effective for treating the brain tumor and/or an additional therapeutic agent in an amount effective for treating the brain tumor.

18. The method of claim 17, wherein said compound, pharmaceutically acceptable salt, or co-crystalline material thereof and said radiation therapy are administered concurrently.

19. The method of claim 17, wherein said compound, pharmaceutically acceptable salt, or cocrystal and said radiation therapy are administered sequentially.

20. The method of claim 16, wherein the brain tumor is characterized by the presence of an IDH1 mutation.

21. The method of claim 20, wherein the IDH1 mutation is an R132X mutation.

22. The method of claim 21, wherein the IDH1 mutation is an R132H or R132C mutation.

23. The method of claim 16, wherein the brain tumor is characterized by the presence of an IDH2 mutation.

24. The method of claim 23, wherein the IDH2 mutation is an R140X or R172X mutation.

25. The method of claim 24, wherein the IDH2 mutation is an R140Q, R140W, or R140L mutation.

26. The method of claim 24, wherein the IDH2 mutation is an R172K or R172G mutation.

27. The method of claim 16, wherein the brain tumor is glioma.
